(19) United States
(12) Patent Application Publication
(10) Pub. No.: US 2017/0159101 A1
(43) Pub. Date: Jun. 8, 2017

(54) DRUG-RESISTANT P97 ATPASE MUTATIONS

(71) Applicant: Cleave Biosciences, Inc., Burlingame, CA (US)

(72) Inventors: Daniel Anderson, El Cerrito, CA (US); Julie Rice, San Francisco, CA (US)

(21) Appl. No.: 15/324,718

(22) PCT Filed: Jul. 9, 2015
(86) PCT No.: PCT/US2015/039749

§ 371 Date: Jan. 8, 2017

Related U.S. Application Data
(60) Provisional application No. 62/022,524, filed on Jul. 9, 2014.

(51) Int. Cl. C12Q 1/34; C12Q 1/68; C12Q 1/70

(52) U.S. Cl. CPC C12Q 1/34; C12Y 306/01003; C12Q 1/701; C12Q 1/6886; C12Q 2600/156; C12Q 2600/106

(57) ABSTRACT

The invention relates to detection of drug insensitivity by detecting mutations in the p97 gene or the p97 gene product following treatment with a p97 inhibitor, and identification of drugs to overcome such drug insensitivity.

### BACKGROUND

Drug resistance is a principal mechanism by which cancers stop responding to chemotherapeutic drugs. It affects patients with a variety of blood cancers and solid tumors, including but not limited to multiple myeloma, neuroedocrine, leukemias, breast, ovarian, lung, and lower gastrointestinal tract cancers. Tumor cells are usually characterized as mutagenic, leading to continued genetic alterations that can render some tumor cells resistant to anti-cancer agents. In addition, tumors may be heterogeneous, with sub-populations of the cancer cells that become drug-resistant. As these resistant cancer cells grow into larger masses following destruction of more sensitive cells in the tumor by an anti-cancer agent, the resistant cells can remain unaffected to additional anti-cancer agent treatment.

Methods for identifying which mutations give rise to drug resistance to drugs, and for identifying drugs that obviate such drug resistance, can facilitate identification of chemotherapeutic agents that will be effective in cancer patients exhibiting drug resistance.

### SUMMARY

The invention relates to drug-resistant cells and p97 ATPases that have been generated in the laboratory, as well as to methods for identification of compounds that can obviate the drug resistance of such drug-resistant cells and p97 ATPases. The invention also relates to methods for identifying in cancer patients genetic mutations in p97 ATPases involved in drug resistance, and developing alternative therapeutic regimens that are effective against the drug-resistant cells. The alternative therapeutic regimen can involve a drug that is a p97 inhibitor and that shows inhibition of p97 activity in cells that are resistant to other p97 inhibitors. Examples of such drugs are described herein.

A first aspect of the invention is a method that involves determining whether a test sample includes:

(i) a mutant p97 polypeptide, or

(ii) a nucleic acid encoding a mutant p97 polypeptide,

wherein the identified, mutant p97 polypeptide includes a sequence with glycine at positions 480 and 481 and at least one amino acid difference compared to a wild type p97, but the p97 polypeptide does not have any of the following mutations: E305Q, E578Q, N348I, N624I, K251A, K524A, R359E, or R635E.

The test sample may be obtained from or may be a mutated laboratory culture of cells useful for determining inhibition of p97 activity or may be from or may be tumor cells from a cancer patient who has become refractory to therapy with a p97 inhibitor drug. Determining whether a test sample includes such amino acid differences can be by nucleic acid sequencing, nucleic acid amplification, reverse transcription, single nucleotide polymorphism assay, primer extension, immunoassay, and/or combinations of such assays or other procedures available to those of skill in the art. Microarrays, dipsticks, and kits are described herein that can be employed for such determinations.

A second aspect of the invention is a method that includes: (i) contacting a drug-resistant cell population with a candidate test compound; and (ii) determining whether the candidate test compound inhibits cell growth of the cell population compared to a control drug-resistant cell population that is not contacted with the candidate test compound.

Such methods are useful for identifying a candidate test compound that can inhibit the ATPase activity and/or cell growth of a drug-resistant cell population. The drug-resistant cell population can express one or more of the mutant p97 polypeptides described herein.

A third aspect of the invention is a method that includes contacting a candidate test compound with such a mutant p97 polypeptide in an in vitro assay or a cellular assay in which the cells contain the mutant p97 polypeptide and determining the in vitro or in vivo ATPase activity of the p97 polypeptide.

A fourth aspect of the invention is a laboratory generated, “mutant” p97 polypeptide having a sequence with at least 95% sequence identity to SEQ ID NO:1, and with at least one amino acid difference compared to a wild type p97 protein. Such p97 polypeptides can be referred to as mutant p97 polypeptides or proteins. Nucleic acids, expression cassettes, expression vectors, and cells expressing such p97 proteins are also described herein, and are part of the invention.

In another aspect of the invention, a mutant p97 polypeptide is identified which includes a sequence with glycine at positions 480 and 481 and at least one amino acid difference compared to a wild type p97, but the p97 polypeptide does not have any of the following mutations: E305Q, E578Q, N348I, N624I, K251A, K524A, R359E, or R635E.

### DESCRIPTION OF THE DRAWING

FIG. 1 is a schematic diagram of the ATPase domains of p97, with domains D1 and D2. The D1 ATPase domain includes amino acid positions from about 209 to about 460. The D2 ATPase domain includes amino acid positions from about 481 to about 763.

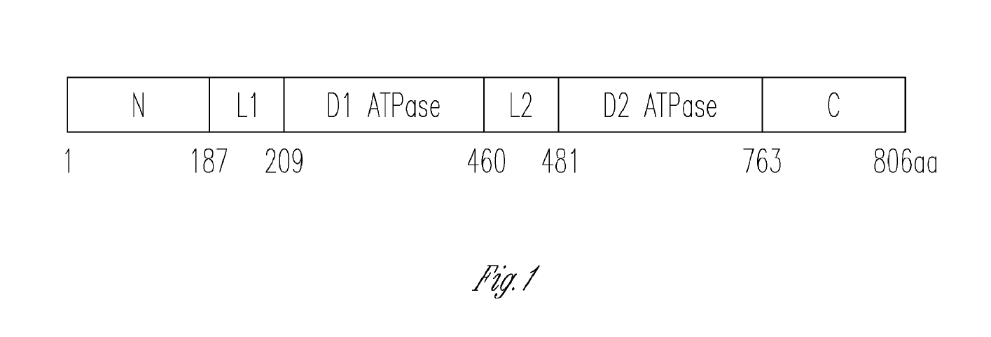

### DETAILED DESCRIPTION

Aspects of the invention relate to identification of the causes of drug insensitivity to p97 inhibitors and methods to obviate such drug insensitivity by identifying which drugs are effective, even against mutant and drug-resistant cells. Drug-insensitive p97 ATPase polypeptides are described herein as well as drug resistant cell lines that express such mutant p97 polypeptides. These drug insensitive p97 ATPase mutants and cell lines were generated in the laboratory and the results indicate that treatment of people with p97 inhibitors may result in the generation of mutant p97 in tumor cells. The in vitro and in vivo activities of these drug insensitive p97 ATPase mutants and cell lines generated in the laboratory in the presence and absence of p97 inhibitors are also described herein. The methods of detecting such drug insensitive p97 ATPase mutants in a patient combined with methods of selecting an effective p97 inhibitor when such drug insensitive p97 ATPase mutants are detected provide a path towards solving intransigent p97 drug insensitivity in patients.

### Methods of Drug Selection

As illustrated herein, different p97 inhibitors are more or less active for inhibition of a specific p97 mutant polypeptide. This is important because p97 inhibitors can be highly effective therapeutic agents for a variety of diseases and conditions (e.g., cancer and viral infections), but over time a subject can become resistant to an administered p97 inhibitor due to mutations in the subject's p97 genes.

Disclosed are assays and techniques for identifying p97 mutations as laboratory embodiments and as developments of patient refractory issues. Also disclosed are assays and techniques for identifying which p97 inhibitors are effective against which p97 mutant polypeptides. Examples of specific p97 inhibitors effective against certain p97 mutant polypeptides are also disclosed. Upon detection of a particular p97 mutation, the methods described herein guide selection of an effective p97 inhibitor for treatment of the subject patient.

“Inhibiting p97” includes affecting any detectable amount of inhibition of p97 activity. A compound can be a p97 inhibitor when the concentration at which the compound inhibits a cell population growth or p97 ATPase activity by at least 5%, or by at least 10%, or by at least 20%, or by at least 25%, or by at least 30%, or by at least 35%, or by at least 40%, or by at least 50%, or by at least 60%, or by at least 70%, or by at least 80%, or by at least 90%, or by at least 95%, or by 95% or greater. A compound can be a p97 inhibitor when the compound inhibits growth of a cell population (e.g., a cancer cell population), or when a p97 inhibitor inhibits the ATPase activity of p97 polypeptide.

When a p97 inhibitor has demonstrated ATPase inhibitory activity against a specific p97 protein (e.g., wild type p97), but a mutant p97 protein is not inhibited by the same p97 inhibitor, that mutant p97 protein is drug insensitive. Subjects expressing mutant p97 proteins in their tumor cells can be resistant to treatment by p97 inhibitors. While it is not a limitation or parameter of the invention, it is believed that such mutations occur in cancer patients because of typical cellular inability to accurately and faithfully reproduce an exact copy of the cell's DNA sequence during cellular replication. This inability is believed to lead to random mutations in proteins coded by the DNA. Some of these mutations may lead to drug resistant p97 enzymatic complexes in particular when the cells and/or organisms and/or patients are under therapeutic stress resulting from administration of p97 inhibitors. The mutations of laboratory cells used in in vivo assays for p97 inhibitory activity described herein demonstrate this random mutation theory incident with inhibitor stress. While a direct correlation of the laboratory mutations of p97 described herein and p97 mutations occurring in cancer patients who would be treated with p97 inhibitors is unknown, it is believed that the laboratory mutations provide insight into which p97 inhibitors may be of benefit to cancer patients whose cancers display resistance toward certain other p97 inhibitors.

Inhibition of p97 can be observed directly or indirectly. Direct inhibition can be observed by observing inhibition of cell growth and/or p97 ATPase activity. However, p97 inhibition includes amelioration of the symptoms of a disease or disorder in which p97 activity is implicated. Such diseases and disorders include cell proliferative disorders, including but not limited to cancer, lysosomal storage diseases, cystic fibrosis, retinitis pigmentosa and viral infections. Inhibition of p97 can include modulation in expression levels of proteins and modifications of proteins such as CHOP, BiP, ATF3, ATF4, p62, NOXA, Nrf1, DRS, poly-ubiquitinated proteins, lysine-48 linked ubiquitin chains, phosphorylated PERK, spliced XBP-1. Hence, p97 inhibition can also be detected by observing amelioration of the symptoms of a disease or disorder in which p97 activity is implicated, or by observing expression of genes or gene products such as CHOP, BiP, ATF3, ATF4, and NOXA, Nrf1, DRS, poly-ubiquitinated proteins, lysine-48 linked ubiquitin chains, phosphorylated PERK, sliced XBP-1, and/or p62.

In some situations, the activity of p97 is measured in vivo within cells, for example, by measuring the growth of cells in the presence and/or absence of a selected p97 inhibitor. The activity of a p97 inhibitor can be reported as the concentration of the p97 inhibitor required for 50% inhibition of cell growth (growth inhibition or GI₅₀).

It is often useful to compare the effectiveness of different p97 inhibitors. When a first p97 inhibitor has a lower GI₅₀ value than a second p97 inhibitor, the first p97 inhibitor has more activity against that cell type than the second p97 inhibitor.

Similarly, it is useful to know which p97 inhibitors are effective against which types of p97 mutations. The GI₅₀ value of a p97 inhibitor in a mutant cell population can therefore be compared to the GI₅₀ value of the same p97 inhibitor in the parental cells from which the mutant cell population was derived (e.g., a wild type parental cell population). Tables of data provided herein report the fold change in GI₅₀ value for each type of p97 inhibitor when measured in a mutant cell line compared to the GI₅₀ value for that p97 inhibitor in the parental (wild type) cell line. A higher fold change in wild type to mutant GI₅₀ value indicates that the p97 inhibitor requires a higher concentration to be effective in the mutant cells than in wild type cells. Conversely, a lower fold change in the wild type to mutant GI₅₀ value indicates that the p97 inhibitor may still retain some efficacy against mutant cells. Thus, when a first p97 inhibitor has been administered to a subject, that first p97 inhibitor may no longer be effective to treat the subject's disease when it exhibits a higher GI₅₀ value than it did against a wild type p97 polypeptide or against a wild type cell population. Upon detection of a higher GI₅₀ value, or upon detection of the p97 mutations described herein, a second p97 inhibitor with a lower GI₅₀ fold change can be administered to the subject, which will have more efficacy against disease provided the pharmacology parameters are similar.

Thus, the difference in fold change of GI₅₀ values between p97 inhibitors are an important selection criteria for identifying a suitable drug for treatment of drug resistant patients. For example, as shown in Table 1, cell line #152 was generated by exposure to the p97 inhibitor described herein as compound 4. Cell line #152 has a p97 mutation that reduces its sensitivity to compound 4, which is reflected in a high GI₅₀ fold change (60.7) in resistance to compound 4 compared to the resistance of the parental cell line to compound 4. This means that about 60 times more compound 4 is needed to inhibit cell growth of cell line 152 than is required for inhibition of the wild type parental cell line. However, another p97 inhibitor described herein, compound 6, has only a 1.42 fold change in GI₅₀ for cell line #152 for when compared to the parental cell line. Therefore compound 6 would be more suitable for treatment of patients who exhibit the mutation present in cell line #152, and/or for treatment of patients who become resistant to compound 4.

In some situations, the activity of p97 is measured by performing in vitro assays of p97 ATPase activity. The activity of a p97 inhibitor can be reported as the concentration of the p97 inhibitor required for 50% inhibition of p97 ATPase activity (IC₅₀). When a first p97 inhibitor has a lower IC₅₀ value than a second p97 inhibitor, the first p97 inhibitor has more activity against p97 protein than the second p97 inhibitor. The IC₅₀ value of a p97 inhibitor against a mutant p97 polypeptide can also be compared to the IC₅₀ value of the same p97 inhibitor against a wild type p97 polypeptide, or against the IC₅₀ value of the same p97 inhibitor against another mutant p97 polypeptide. In general, p97 inhibitors with lower IC₅₀ values are more active. Thus, for example, when a first p97 inhibitor has been administered to a subject, and that first p97 inhibitor is no longer effective to inhibit p97 activity, the subject may express a p97 mutant protein. The application describes which type of p97 inhibitor is more effective for inhibition of which type of mutant p97 polypeptide. Thus, pursuant to the methods described herein better p97 inhibitors can be selected when specific p97 mutations are detected in sample obtained from a subject, e.g., a patient, suspected of being resistant to an administered drug.

### p97 Structures and Activities

Mutant p97 genes were generated in the laboratory from parental cell lines that were homozygous for the wild type (non-mutant) p97 gene. To generate the clonal parental cell lines in the experiments described herein, single cells were selected from Hct116, DLD-1, or LoVo cell lines. These cell lines are all human colon carcinoma cell lines commercially available from Sigma Aldrich. Hence, the wild type p97 polypeptide discussed herein is human p97 and the mutant p97 polypeptides are mutant human p97 polypeptides.

These cell lines were grown to form a clonal population of cells; such a clonal population is a parental cell line. The parental cell lines were each sequenced to insure that no p97 mutations were present. Hence, the parental cell lines had a wild type p97 gene, and expressed a wild type p97 polypeptide. Different aliquots of these parental cells were then exposed to different p97 inhibitors, separate resistant cells were identified and cloned, and then the sequences of the p97 genes in these distinct p97 inhibitor resistant clonal cell lines were determined. The p97 mutations and mutant p97 polypeptides were therefore generated in the laboratory.

As explained above, the p97 inhibitor compounds did not and do not directly cause mutations in the cellular genetic code. Instead, mutations occur randomly because of imperfect replication of the cellular genetic code during cellular division and replication. The presence of the p97 inhibitor compounds enables certain mutant cells to continue replication while non-mutated cells wither away. The result is natural selection of the drug resistant mutant cells.

The p97 protein has three domains (N-domain, D1 ATPase domain, and D2 ATPase domain) joined together by linker regions. X-ray crystallography of p97 revealed that it forms a homohexamer of 97 kilodalton subunits that assemble to form two stacked rings. The two rings are formed by the ATPase domains (Huyton, T. et al. Jan. 16, 2009. Struct. Biol. (2003) 144, 337-348; DeLaBarre, B. et al. Nat. Struct. Biol. (2003) 10, 856-863). The ‘top’ ring is formed by a hexamer of the D1 domains, and the ‘bottom’ ring is formed by a hexamer of the D2 domains. The N-domain extends outward from the D1 domain ring.

A sequence for the human p97 protein is available from the National Center for Biotechnology Information (NCBI) (see website at ncbi.nlm.nih.gov) as accession number NP_009057.1 (GI: 6005942) and provided below as SEQ ID NO:1.

| | |
|---|---|
| 1 | MASGADSKGD DLSTAILKQK NRPNRLIVDE AINEDNSVVS |
| 41 | LSQPKMDELQ LFRGDTVLLK GKKRREAVCI VLSDDTCSDE |
| 81 | KIRMNRVVRN NLRVRLGDVI SIQPCPDVKY GKRIHVLPID |
| 121 | DTVEGITGNL FEVYLKPYFL EAYRPIRKGD IFLVRGGMRA |
| 161 | VEFKVVETDP SPYCIVAPDT VIHCEGEPIK REDEEESLNE |
| 201 | VGYDDIGGCR KQLAQIKEMV ELPLRHPALF KAIGVKPPRG |
| 241 | ILLYGPPGTG KTLIARAVAN ETGAFFFLIN GPEIMSKLAG |
| 281 | ESESNLRKAF EEAEKNAPAI IFIDELDAIA PKREKTHGEV |
| 321 | ERRIVSQLLT LMDGLKQRAH VIVMAATNRP NSIDPALRRF |
| 361 | GRFDREVDIG IPDATGRLEI LQIHTKNMKL ADDVDLEQVA |
| 401 | NETHGHVGAD LAALCSEAAL QAIRKKMDLI DLEDETIDAE |
| 441 | VMNSLAVTMD DFRWALSQSN PSALRETVVE VPQVTWEDIG |
| 481 | GLEDVKRELQ ELVQYPVEHP DKFLKFGMTP SKGVLFYGPP |
| 521 | GCGKTLLAKA IANECQANFI SIKGPELLTM WFGESEANVR |
| 561 | EIFDKARQAA PCVLFFDELD SIAKARGGNI GDGGGAADRV |
| 601 | INQILTEMDG MSTKKNVFII GATNRPDIID PAILRPGRLD |
| 641 | QLIYIPLPDE KSRVAILKAN LRKSPVAKDV DLEFLAKMTN |
| 681 | GFSGADLTEI CQRACKLAIR ESIESEIRRE RERQTNPSAM |
| 721 | EVEEDDPVPE IRRDHFEEAM RFARRSVSDN DIRKYEMFAQ |
| 761 | TLQQSRGFGS FRFPSGNQGG AGPSQGSGGG TGGSVYTEDN |
| 801 | DDDLYG |

The p97 amino acid sequence provided above as SEQ ID NO:1 is encoded by a cDNA with the following sequence (NCBI accession number NM_007126.3, GI:169881236; SEQ ID NO:2).

| | |
|---|---|
| 1 | GTGATCTGCG GGTTGCTGGG GAGAGGCGCG GAGAGGCGGG |
| 41 | CGAGAGTCCG CAGGGCAGGC GCTGATTGGC TGAGGTGGGA |
| 81 | GCAGCTTCCC TTCCGATGAT TCGGCTCTTC TCGGCTCAGT |
| 121 | CTCAGCGAAG CGTCTGCGAC CGTCGTTTGA GTCGTCGCTG |
| 161 | CCGCTGCCGC TGCCACTGCC ACTGCCACCT CGCGGATCAG |
| 201 | GAGCCAGCGT TGTTCGCCCG ACGCCTCGCT GCCGGTGGGA |
| 241 | GGAAGCGAGA GGGAAGCCGC TTGCGGGTTT GTCGCCGCTG |
| 281 | CTCGCCCACC GCCTGGAAGA GCCGAGCCCC GGCCCAGTCG |
| 321 | GTCGCTTGCC ACCGCTCGTA GCCGTTACCC GCGGGCCGCC |
| 361 | ACAGCCGCCG GCCGGGAGAG GCGCGCGCCA TGGCTTCTGG |
| 401 | AGCCGATTCA AAAGGTGATG ACCTATCAAC AGCCATTCTC |
| 441 | AAACAGAAGA ACCGTCCCAA TCGGTTAATT GTTGATGAAG |
| 481 | CCATCAATGA GGACAACAGT GTGGTGTCCT TGTCCCAGCC |
| 521 | CAAGATGGAT GAATTGCAGT TGTTCCGAGG TGACACAGTG |
| 561 | TTGCTGAAAG GAAAGAAGAG ACGAGAAGCT GTTTGCATCG |
| 601 | TCCTTTCTGA TGATACTTGT TCTGATGAGA AGATTCGGAT |
| 641 | GAATAGAGTT GTTCGGAATA ACCTTCGTGT ACGCCTAGGG |
| 681 | GATGTCATCA GCATCCAGCC ATGCCCTGAT GTGAAGTACG |
| 721 | GCAAACGTAT CCATGTGCTG CCCATTGATG ACACAGTGGA |
| 761 | AGGCATTACT GGTAATCTCT TCGAGGTATA CCTTAAGCCG |
| 801 | TACTTCCTGG AAGCGTATCG ACCCATCCGG AAAGGAGACA |
| 841 | TTTTTCTTGT CCGTGGTGGG ATGCGTGCTG TGGAGTTCAA |
| 881 | AGTGGTGGAA ACAGATCCTA GCCCTTATTG CATTGTTGCT |
| 921 | CCAGACACAG TGATCCACTG CGAAGGGGAG CCTATCAAAC |
| 961 | GAGAGGATGA GGAAGAGTCC TTGAATGAAG TAGGGTATGA |
| 1001 | TGACATTGGT GGCTGCAGGA AGCAGCTAGC TCAGATAAAG |
| 1041 | GAGATGGTGG AACTGCCCCT GAGACATCCT GCCCTCTTTA |
| 1081 | AGGCAATTGG TGTGAAGCCT CCTAGAGGAA TCCTGCTTTA |
| 1121 | CGGACCTCCT GGAACAGGAA AGACCCTGAT TGCTCGAGCT |
| 1161 | GTAGCAAATG AGACTGGAGC CTTCTTCTTC TTGATCAATG |
| 1201 | GTCCTGAGAT CATGAGCAAA TTGGCTGGTG AGTCTGAGAG |
| 1241 | CAACCTTCGT AAAGCCTTTG AGGAGGCTGA GAAGAATGCT |
| 1281 | CCTGCCATCA TCTTCATTGA TGAGCTAGAT GCCATCGCTC |
| 1321 | CCAAAAGAGA GAAAACTCAT GGCGAGGTGG AGCGGCGCAT |
| 1361 | TGTATCACAG TTGTTGACCC TCATGGATGG CCTAAAGCAG |
| 1401 | AGGGCACATG TGATTGTTAT GGCAGCAACC AACAGACCCA |
| 1441 | ACAGCATTGA CCCAGCTCTA CGGCGATTTG GTCGCTTTGA |
| 1481 | CAGGGAGGTA GATATTGGAA TTCCTGATGC TACAGGACGC |
| 1521 | TTAGAGATTC TTCAGATCCA TACCAAGAAC ATGAAGCTGG |
| 1561 | CAGATGATGT GGACCTGGAA CAGGTAGCCA ATGAGACTCA |
| 1601 | CGGGCATGTG GGTGCTGACT TAGCAGCCCT GTGCTCAGAG |
| 1641 | GCTGCTCTGC AAGCCATCCG CAAGAAGATG GATCTCATTG |
| 1681 | ACCTAGAGGA TGAGACCATT GATGCCGAGG TCATGAACTC |
| 1721 | TCTAGCAGTT ACTATGGATG ACTTCCGGTG GGCCTTGAGC |
| 1761 | CAGAGTAACC CATCAGCACT GCGGGAAACC GTGGTAGAGG |
| 1801 | TGCCACAGGT AACCTGGGAA GACATCGGGG GCCTAGAGGA |
| 1841 | TGTCAAACGT GAGCTACAGG AGCTGGTCCA GTATCCTGTG |
| 1881 | GAGCACCCAG ACAAATTCCT GAAGTTTGGC ATGACACCTT |
| 1921 | CCAAGGGAGT TCTGTTCTAT GGACCTCCTG GCTGTGGGAA |
| 1961 | AACTTTGTTG GCCAAAGCCA TTGCTAATGA ATGCCAGGCC |
| 2001 | AACTTCATCT CCATCAAGGG TCCTGAGCTG CTCACCATGT |
| 2041 | GGTTTGGGGA GTCTGAGGCC AATGTCAGAG AAATCTTTGA |
| 2081 | CAAGGCCCGC CAAGCTGCCC CCTGTGTGCT ATTCTTTGAT |
| 2121 | GAGCTGGATT CGATTGCCAA GGCTCGTGGA GGTAACATTG |
| 2161 | GAGATGGTGG TGGGGCTGCT GACCGAGTCA TCAACCAGAT |
| 2201 | CCTGACAGAA ATGGATGGCA TGTCCACAAA AAAAAATGTG |
| 2241 | TTCATCATTG GCGCTACCAA CCGGCCTGAC ATCATTGATC |
| 2281 | CTGCCATCCT CAGACCTGGC CGTCTTGATC AGCTCATCTA |
| 2321 | CATCCCACTT CCTGATGAGA AGTCCCGTGT TGCCATCCTC |
| 2361 | AAGGCTAACC TGCGCAAGTC CCCAGTTGCC AAGGATGTGG |
| 2401 | ACTTGGAGTT CCTGGCTAAA ATGACTAATG GCTTCTCTGG |
| 2441 | AGCTGACCTG ACAGAGATTT GCCAGCGTGC TTGCAAGCTG |
| 2481 | GCCATCCGTG AATCCATCGA GAGTGAGATT AGGCGAGAAC |
| 2521 | GAGAGAGGCA GACAAACCCA TCAGCCATGG AGGTAGAAGA |
| 2561 | GGATGATCCA GTGCCTGAGA TCCGTCGAGA TCACTTTGAA |
| 2601 | GAAGCCATGC GCTTTGCGCG CCGTTCTGTC AGTGACAATG |
| 2641 | ACATTCGGAA GTATGAGATG TTTGCCCAGA CCCTTCAGCA |
| 2681 | GAGTCGGGGC TTTGGCAGCT TCAGATTCCC TTCAGGGAAC |
| 2721 | CAGGGTGGAG CTGGCCCCAG TCAGGGCAGT GGAGGCGGCA |
| 2761 | CAGGTGGCAG TGTATACACA GAAGACAATG ATGATGACCT |
| 2801 | GTATGGCTAA GTGGTGGTGG CCAGCGTGCA GTGAGCTGGC |
| 2841 | CTGCCTGGAC CTTGTTCCCT GGGGGTGGGG GCGCTTGCCC |
| 2881 | AGGAGAGGGA CCAGGGGTGC GCCCACAGCC TGCTCCATTC |
| 2921 | TCCAGTCTGA ACAGTTCAGC TACAGTCTGA CTCTGGACAG |
| 2961 | GGGGTTTCTG TTGCAAAAAT ACAAAACAAA AGCGATAAAA |
| 3001 | TAAAAGCGAT TTTCATTTGG TAGGCGGAGA GTGAATTACC |
| 3041 | AACAGGGAAT TGGGCCTTGG GCCTATGCCA TTTCTGTTGT |
| 3081 | AGTTTGGGGC AGTGCAGGGG ACCTGTGTGG GGTGTGAACC |
| 3121 | AAGGCACTAC TGCCACCTGC CACAGTAAAG CATCTGCACT |
| 3161 | TGACTCAATG CTGCCCGAGC CCTCCCTTCC CCCTATCCAA |
| 3201 | CCTGGGTAGG TGGGTAGGGG CCACAGTTGC TGGATGTTTA |
| 3241 | TATAGAGAGT AGGTTGATTT ATTTTACATG CTTTTGAGTT |
| 3281 | AATGTTGGAA AACTAATCAC AAGCAGTTTC TAAACCAAAA |
| 3321 | AATGACATGT TGTAAAAGGA CAATAAACGT TGGGTCAAAA |
| 3361 | TGGAGCCTGA GTCCTGGGCC CTGTGCCTGC TTCTTTTCCT |
| 3401 | GGGAACAGCC TTGGGCTACC CACCACTCCC AAGGCATTCT |
| 3441 | TCCAAATGTG AAATCCTGGA AGTAAGATTG CACCTTCTTC |
| 3481 | CTCTCCTGAT CAACATCGGT ATGATGTCTC CTGTTGCCTC |
| 3521 | ACCCTTTGTC TGCAGTATCA CTGGATAGGA CTGGTGGAAA |
| 3561 | GGGAGCAGCC TGACAGAGCT CCAAATGTGG AGAATATGGC |
| 3601 | ATCCCTCCAC CTATATTTGA TGTGGACGGT AAGGCTAGGC |
| 3641 | CTGCAGGATC CCTTATCCTG ACCAAAGACT GTGTTGGGGT |
| 3681 | GCCATTTGAA AATCGCAGGG TTGCAAAAGA ATACAATCTT |
| 3721 | ACTTGCAGGT GGATATTCTC TATACTCTCT TTTAATGCAT |
| 3761 | CTAAAAATCC CAAACATCCC CTGGTTGGTG ATCACTTACA |
| 3801 | GTTGTGTCCA CCTTTATTTT ATGTACTTTG ATTAAAAAAA |
| 3841 | AAAAACTTTT TGTTAATAT |

As described herein, mutations that give rise to resistance against p97 inhibitors are substantially all within the D2 domain of the p97 protein. The D2 domain of the p97 protein includes a polypeptide segment from about position 481 to about position 763 of the SEQ ID NO:1 p97 protein. The sequence of this D2 domain is shown below as SEQ ID NO: 3.

| | |
|---|---|
| 481 | GLEDVKRELQ ELVQYPVEHP DKFLKFGMTP SKGVLFYGPP |
| 521 | GCGKTLLAKA IANECQANFI SIKGPELLTM WFGESEANVR |
| 561 | EIFDKARQAA PCVLFFDELD SIAKARGGNI GDGGGAADRV |
| 601 | INQILTEMDG MSTKKNVFII GATNRPDIID PAILRPGRLD |
| 641 | QLIYIPLPDE KSRVAILKAN LRKSPVAKDV DLEFLAKMTN |
| 681 | GFSGADLTEI CQRACKLAIR ESIESEIRRE RERQTNPSAM |
| 721 | EVEEDDPVPE IRRDHFEEAM RFARRSVSDN DIRKYEMFAQ |
| 761 | TLQ |

The D2 domain can have one or more amino acid substitutions, deletions, or additions.

For example, the region of p97 that can have mutations can be within the p97 segment shown below as SEQ ID NO:4.

| | |
|---|---|
| 481 | GLEDVKRELQ ELVQYPVEHP DKFLKFGMTP SKGVLFYGPP |
| 521 | GCGKTLLAKA IANECQANFI SIKGPELLTM WFGESEANVR |
| 561 | EIFDKARQAA PCVLFFDELD SIAKARGGNI GDGGGAADRV |
| 601 | INQILTEMDG MSTKKNVFII GATNRPDIID PAILRPGRLD |
| 641 | QLIYIPLPDE KSRVAILKAN LRKSPVAKDV DLEFLAKMTN |
| 681 | GFSGADLTEI |

Moreover, mutations correlating with resistance against p97 ATP-competitive inhibitors tend to cluster within various regions of the D2 domain. For example, mutations tend to cluster within a segments of the p97 D2 domain from about amino acid position 649 to about position 688, shown below as SEQ ID NO:5.

| | |
|---|---|
| 649 | DE KSRVAILKAN LRKSPVAKDV DLEFLAKMTN |
| 681 | GFSGADLT |

Another segment of the p97 protein that correlates with resistance to p97 inhibitors is with the D2 domain from amino acid position 470 to about position 535, shown below as SEQ ID NO:6.

| | |
|---|---|
| 470 | E VPQVTWEDIG |
| 481 | GLEDVKRELQ ELVQYPVEHP DKFLKFGMTP SKGVLFYGPP |
| 521 | GKTLLAKAIA NEC |

Although many mutations present in the mutant p97 polypeptides described herein are within the D2 domain, the mutant p97 polypeptides described herein may also have mutations outside of the D2 domain. For example, the mutant p97 polypeptides described herein also have mutations at amino acid positions that are within about position 454 to about position 550 of the D2 domain, or at any position within 15 amino acids of the D2 domain.

Examples of mutant p97 polypeptides include those with mutations at one or more of the following amino acid positions: E470, P472, Q473, V474, T475, G481, L482, V485, E498, P500, F516, A528, C535, F539, S541, A569, I620, D649, D649, A659, N660, N660, T688, or any combination thereof.

Specific mutations that correlate with resistance against p97 inhibitors include the following: E470D, E470E/D, P472A, P472L, P472S, Q473P, V474A, V474V/A, T475I, G481A, L482I, V485D, E498D, P500T, F516L, A528T, C535Y, F539I, S541P, A569A/T, A569T, I620I/L, P646T, D649A, D649D/N, D649N, A659T, N660D, N660K, N660N/D, N660N/S, T688A, T688I, and combinations thereof.

In some embodiments, the mutant p97 polypeptide has glycine at positions 480 and 481 as is present in wild type p97 polypeptide but will have a mutation elsewhere, especially as listed above. In some embodiments, the mutant p97 polypeptide does not have glutamine at position 578. In some embodiments, the p97 mutation does not include alteration of a cysteine at position 522. In other words, the mutant p97 polypeptides described herein can have a cysteine at position 522, but have at least one other mutation in its amino acid sequence. In some embodiments, the mutant p97 polypeptides described herein do not have alanine at position 522. In other words, the mutant p97 polypeptides described herein can have an amino acid other than cysteine at position 522, but that amino acid is not an alanine.

The nucleotide and amino acid positions described herein are relative to the sequences (SEQ ID NOs) described herein, but the same mutated positions are present in p97 nucleic acids and proteins that may somewhat different position numbering (e.g., due to additional or fewer nucleotides or amino acids at the 5′ end of the p97 cDNA or at the N-terminus of the p97 polypeptide).

The mutations in the p97 ATPases can affect p97 function in a variety of ways. ATPase p97 is conserved across all eukaryotes and is essential for life in budding yeast (Giaever, G., et. al. Nature (2002) 418, 387-391) and mice (Muller, J. M. et al. Biochem. Biophys. Res. Commun. (2007) 354, 459-465). Loss-of-function studies in model organisms indicate that p97 plays a critical role in a broad array of cellular processes including Golgi membrane reassembly (Rabouille, C. et al. Cell (1995) 82, 905-914), membrane transport (Ye, Y. et al Nature (2001) 414, 652-656; Ye, Y. et al. Nature (2004) 429, 841-847) degradation of misfolded membrane and secretory proteins by the ubiquitin-proteasome system (UPS) (Golbik, R. et al. Biol. Chem. (1999) 380, 1049-1062; Richly, H. et al. Cell (2005) 120, 73-84), regulation of myofibril assembly (Janiesch, P. C. et al. Nat. Cell Biol. (2007) 9, 379-390), and cell division (Cao, K. et al. Cell (2003) 115, 355-367). Humans bearing reduction-of-cellular function alleles of p97 are afflicted with a syndrome that includes inclusion body myopathy and frontotemporal lobar degeneration (Weihl, C. et al. Hum. Mol. Genet. (2006) 15, 189-199).

Studies indicate that the p97 proteins unfold proteins or disassemble protein complexes. The activity of p97 is linked to substrate proteins by an array of at least 14 ubiquitin regulatory X (UBX) domain-containing proteins that bind p9′7, as well as the non-UBX domain adaptors Ufd1 and Npl4 (Meyer, H. H. et al. EMBO J. (2000) 19, 2181-2192).

Although the D2 domain of p97 hydrolyzes ATP in vitro, the level of D1-specific ATPase activity reported by different investigators varies. Genetic studies in yeast indicate that p97 function may require ATP hydrolysis by both the D1 and D2 domains (Song, C. et al. J. Biol. Chem. (2003) 278, 3648-3655; Ye, Y. et al. J. Cell Biol. (2004) 162, 71-84). Binding of ATP to the D1 domain is also required for assembly of p97 (Wang, Q. et al. Biochem. Biophys. Res. Commun. (2003) 300, 253-260). Although ATP hydrolysis by the D2 domain is apparently not required for assembly of p97 hexamer, it is thought that ATP hydrolysis by the D2 domain is an obligate step in the catalytic cycle of p97. Studies indicate that ATP hydrolysis by the D2 domain contributes to structural transformations in bound substrates, resulting in their unfolding or dissociation from bound partners.

One role for p97 is in the turnover of misfolded secretory proteins via the ubiquitin-proteasome system (UPS) and the endoplasmic reticulum-associated degradation (ERAD) pathway. Proteins that fail to fold within the endoplasmic reticulum are retro-translocated in a p97-dependent manner into the cytoplasm where they are degraded by the UPS (Ye, Y. et al. Nature (2004) 429, 841-847). In this process, p97 is thought to mediate extraction of substrates from the endoplasmic reticulum membrane. p97 is also required for the turnover of cytosolic substrates of the UPS (Janiesch, P. C. et al. Nat. Cell Biol. (2007) 9, 379-390; Cao, K. et al. Cell (2003) 115, 355-367; Fu, X. et al. J. Cell Biol. (2003) 163, 21-26), although its role in turnover of cytosolic proteins is less understood.

The p97 protein is overproduced in some cancer types (Yamamoto, S. et al. Ann. Surg. Oncol. (2005) 12, 925-934; Yamamoto, S. et al. Clin. Cancer Res. (2004) 10, 5558-5565; Yamamoto, S. et al. Ann. Surg. Oncol. (2004) 11, 697-704; Yamamoto, S. et al. Ann. Surg. Oncol. (2004) 11, 165-172). Studies also indicate that p97 is needed for endoplasmic reticulum-associated protein degradation (ERAD) (Carvalho, P. et al. Cell (2006) 126, 361-373), and that cancer cells may be particularly dependent upon ERAD (Boelens, J. et al. In Vivo (2007) 21, 215-226). Furthermore, p97 has been linked to the turnover of IkB and consequent activation of NF-kB (Dai, R. M. et al. J. Biol. Chem. (1998) 273, 3562-3573). NF-kB activity is important for the survival of some tumor cells, particularly in multiple myeloma (Keats, J. J. et. al. Cancer Cell (2007) 12, 131-144; Annunziata, C. M. et. al. Cancer Cell (2007) 12, 115-130).

Current theory for cancer treatment involving p97 holds that ameliorating the activity of the p97 enzyme complex results in an increase of poly-ubiquitinated protein and ERAD substrates in the cell. This increase signals activation of the apoptosis mechanism leading to cellular death. Hence, inhibition of the p97 enzyme complex leads to death of the cancer cell. In contrast, an active p97 enzymatic pathway avoids activation of apoptosis by translocating and releasing the poly-ubiquitinated proteins to the UPS mechanism which degrades such proteins to constituent amino acids ready for reuse in protein biosynthesis. The active pathway assures continued viability of the cancer cell.

### Drug Insensitive Cell Lines

The mutant p97 nucleic acids and mutant p97 polypeptides described herein were generated by exposing a wild type population of cells to p97 inhibitors in the laboratory. The mechanism for mutant generation is believed to occur as described above. Such drug insensitive cell lines are useful for identifying what mutations are correlated with drug insensitivity. Generation and detection of such mutations by the methods described herein can facilitate identification of drugs that are effective for treatment of drug insensitive subjects.

Additional drug insensitive cell lines can be generated using the methods described herein. For example, a population of cells can be contacted or exposed to selected p97 inhibitors for a time and at a concentration sufficient to generate at least one drug-insensitive cell. Cells that survive and/or replicate in the presence of a selected p97 inhibitor are drug-resistant cells (also called drug-insensitive cells). The drug-insensitive cell(s) can be isolated and cultured to generate separate drug-insensitive cell lines.

The population of cells has a nucleic acid that encodes and/or expresses a p97 wild type or mutant polypeptide (either homozygous or heterozygous). The population of cells can include prokaryotic or eukaryotic cells. In some instances, the population of cells is a population of eukaryotic cells. For example, the population of cells can be primary or established cancer cell lines. The population of cancer cells can include human cancer cells from a solid tumor, a multiple myeloma, a metastatic breast cancer or tumor, a non-small cell lung cancer or tumor, a prostate cancer or tumor, an advanced colorectal cancer or tumor, an ovarian cancer or tumor, primary peritoneal carcinoma, a hormone refractory prostate cancer or tumor, a squamous cell carcinoma of the head, a squamous cell carcinoma of the neck, a metastatic pancreatic adenocarcinoma, a gastroesophageal cancer or tumor, a gastrointestinal cancer or tumor, a stomach cancer or tumor, a leukemia, a non-Hodgkin's lymphoma, or combinations thereof.

Examples of such human cell populations include an Hct116, DLD-1, or LoVo cell line. These cell lines are deficient in mismatch repair and have low expression of multidrug-resistant (MDR) pumps. Therefore, Hct116, DLD-1, and LoVo cell lines will facilitate the rapid identification of mutations that confer drug resistance. These cell lines are commercially available, for example, from the American Type Culture Collection, SigmaAldrich, and Life Technologies. The population of cells can also be an established cancer cell line such as any of those available from a cell depository such as the American Type Culture Collection.

The population of p97-expressing cells will range from about 10³ cells to about 10¹¹ cells, preferably about 10⁵ cells to about 10⁹ cells, and most preferably about 10⁶ cells to about 10⁷ cells, can be contacted (e.g., cultured) in presence of a selected p97 inhibitor.

The population of cells can be incubated in the presence of a selected or candidate p97 inhibitor for at least one cellular replication cycle, or at least two cellular replication cycles, or at least three cellular replication cycles. For example, the population of cells can be incubated with a selected p97 inhibitor for at least about 1 hour, or at least about 4 hours, or at least about 8 hours, or at least about 10 hours, or at least about 12 hours, or at least about 14 hours, or for at least one day. In some instances, the population of cells will be incubated with a selected p97 inhibitor for at least two days, or at least three days, or at least four days, or for at least one week.

The amount of selected p97 inhibitors useful for generating drug-insensitive cells can be determined by plating the cells in a variety of drug concentrations. For example, similar numbers of cells can be plated in various dilutions of the selected p97 inhibitors.

For example, the population of cells can be incubated with about 1.0 nM to about 100 μM of the selected p97 inhibitor(s). In some instances the population of cells can be incubated with 10-point 2-fold serial dilutions centered around the IC₅₀. In some embodiments, the population of cells can be incubated with about 10 nM to about 1 μM, or about 20 nM to about 800 nM, or about 30 nM to about 600 nM, or about 50 nM to about 500 nM, or about 75 nM to about 400 nM, or about 100 nM to about 300 nM of the selected p97 inhibitor(s).

Drug insensitive cells can be identified as small colonies of live cells that survive exposure to the selected p97 inhibitor(s) for the above-indicated times and in the above-indicated concentrations of the selected p97 inhibitor(s).

The drug resistance of different cell lines can be compared by determining the drug dosages of the various cell lines that decrease the viability of the cells by 50% (GI₅₀). Data relating to cell viability versus drug concentration can be plotted to determine compound doses that result in 50% decrease in viability (GI₅₀).

The characteristics of the newly generated drug-resistant cell lines can be compared to the parental cell lines from which they were derived, to wild type cells, and/or to other drug-resistant cell lines. The fold difference in drug sensitivity (e.g., in GI₅₀ values) can be calculated as a measure of the difference between cell lines.

Nucleic acids encoding the drug resistant cells' p97 genes can be sequenced to identify mutation sites correlated with drug resistance. For example, the p97 coding region and/or p97 regulatory elements can be sequenced to identify structural changes in the encoded p97 protein, and/or sites that regulate p97 expression. Examples of primers that can be used are described in Example 1. The nucleic acid that is sequenced can be a genomic DNA, a cDNA, or an mRNA. The primer(s) can include a nucleic acid segment that binds to the p97 nucleic acid with specificity and other components that are not naturally linked to such a segment. For example, the primer(s) can have one or more labels, restriction sites, 5′ extensions, 3′ extensions, or a combination thereof (each of which is not naturally present in the mammalian genome).

### In Vitro Assays

The activities of p97 proteins that are described herein (i.e., both wild type and mutant p97 polypeptides) can be evaluated to ascertain whether they are active in the presence of p97 inhibitors, and to identify which p97 inhibitors are effective against which p97 polypeptide. A variety of assays can be used to evaluate the binding and inhibitory effects of p97 inhibitors to wild type and/or mutant p97 proteins. Such assays can include labeled in vitro protein binding assays, signaling assays using detectable molecules, and ATPase assays. Such assays can be used to rapidly examine the binding of p97 inhibitors, and their effects on the activities of wild type and mutant p97 proteins.

An exemplary assay involves contacting a wild type or mutant p97 protein with a candidate compound (e.g., a candidate p97 inhibitor) under conditions where binding can occur. The candidate compound can be a known p97 inhibitor (e.g., N2,N4-dibenzylquinazoline-2,4-diamine (DBeQ)), or any of the p97 inhibitors described herein.

Binding of an inhibitor to p97 can be detected by retention of the candidate compound by the wild type or mutant p97 protein. Alternatively, binding can be detected by altered ATPase activity of the mutant p97 protein and/or wild type p97 protein. The p97 proteins have ATPase activities where ATP is hydrolyzed to ADP and inorganic phosphate. For example, increased ADP or inorganic phosphate levels in the presence of the candidate compound indicates that the wild type or mutant p97 proteins are active and that the candidate compound is not a particularly effective inhibitor of the p97 ATPase. When no significant change in ATP levels is observed (i.e., little ADP or inorganic phosphate is formed), the candidate compound is an inhibitor of wild type and mutant p97 proteins.

A mutant p97 protein is generally drug insensitive when a p97 inhibitor does not inhibit the activity of this mutant p97 protein, but the p97 inhibitor does inhibit the activity of the wild type p97 protein. A mutant p97 protein may therefore not bind a specific p97 inhibitor as well as that p97 inhibitor is bound by the wild type p97 protein, so that the ATPase activity of mutant p97 protein is higher than that of the wild type p97 protein in the presence of that p97 inhibitor. For example, the mutant p97 polypeptide may have at least 20%, or by at least 25%, or by at least 30%, or by at least 35%, or by at least 40%, or by at least 50%, or by at least 60%, or by at least 70%, or by at least 80%, or by at least 90%, or by at least 100% more ATPase activity than the wild type p97 protein in the presence of a particular p97 inhibitor. In some instance, the mutant p97 polypeptide may have at least two-fold, or at least three-fold, or at least five-fold, or at least ten-fold more ATPase activity than the wild type p97 protein in the presence of a particular p97 inhibitor.

Wild type and mutant p97 proteins used in the methods of the invention can be added to an assay mixture as isolated polypeptides (where binding of a candidate p97 inhibitor is to be measured) or as a cell or other membrane-encapsulated space which includes the wild type or mutant p97 protein. In the latter assay configuration, the cell or other membrane-encapsulated space can contain the wild type and mutant p97 proteins (e.g., a cell transfected with an expression vector containing a wild type or mutant p97 protein). In the assays described herein, the wild type and mutant p97 proteins can be produced recombinantly, isolated from biological extracts, or synthesized in vitro. Mutant p97 proteins encompass chimeric proteins comprising a fusion of a wild type or mutant p97 polypeptide with another polypeptide, e.g., a polypeptide capable of providing or enhancing protein-protein binding, enhancing signaling capability, facilitating detection, or enhancing stability of the wild type or mutant p97 protein under assay conditions. A polypeptide fused to the wild type or mutant p97 polypeptide or fragment thereof may also provide means of readily detecting the fusion protein, e.g., by immunological recognition or by fluorescent labeling.

ATPase assays can be performed in a mixture containing a mutant or wild type p97 protein. A candidate p97 inhibitor can also be present. A variety of other reagents also can be included in the mixture. These include reagents such as salts, buffers, neutral proteins (e.g., albumin), detergents, etc. which may be used to facilitate optimal protein-protein binding. Such a reagent may also reduce non-specific or background interactions of the reaction components. Other reagents that improve the efficiency of the assay such as protease inhibitors, nuclease inhibitors, antimicrobial agents, and the like may also be used. In some instances the assay can contain a buffer such as a Tris-HCl buffer at about pH 7.0 to 7.6. ATP and magnesium can also be present in the assay mixture. A chelating agent can be present as well.

For example, one type of buffer that can be employed contains 50 mM Tris-HCl (pH 7.4), 20 mM MgCl2, 0.1 mM EDTA, 80 mM NaCl, 0.5 mM ATP, and a wild type or mutant p97 protein. The release of inorganic phosphate by ATP hydrolysis can measured using a colorimetric ATPase assay kit (Innova Biosciences). The ATP and the wild type or mutant p97 protein are present in known amounts.

The order of addition of components, incubation temperature, time of incubation, and other parameters of the assay may be readily determined. Such experimentation merely involves optimization of the assay parameters, not the fundamental composition of the assay. Incubation temperatures typically are between 4° C. and 40° C. Incubation times preferably are minimized to facilitate rapid, high throughput screening, and typically are between 1 minute and 10 hours.

After incubation, the level of signaling or the level of specific binding between the mutant or wild type p97 protein and a candidate p97 inhibitor is detected by any convenient method available to the user. For cell-free binding type assays, a separation step can be used to separate bound from unbound components. The separation step may be accomplished in a variety of ways. For example, separation can be accomplished in solution, or, conveniently, at least one of the components is immobilized on a solid substrate, from which the unbound components may be easily separated. For example, the mutant or wild type p97 protein can be bound to a solid support while the candidate compound(s) (e.g., potential p97 inhibitor(s)) are in solution during incubation. Alternatively, the candidate compounds can be tethered to a solid support in different locations, and the mutant or wild type p97 protein can be in solution during incubation. After incubation unbound components can be washed away and a complex formed between a p97 protein and a compound can be detected by available procedures. Binding indicates that the compound so bound can be a p97 inhibitor.

The solid substrate can be made of a wide variety of materials and in a wide variety of shapes, e.g., microtiter plate, microbead, dipstick, resin particle, etc. The substrate can be chosen to maximize signal-to-noise ratios, primarily to minimize background binding, as well as for ease of separation and cost. Separation may be effected for example, by removing a bead or dipstick from a reservoir, emptying or diluting a reservoir such as a microtiter plate well, rinsing a bead, particle, chromatographic column or filter with a wash solution or solvent. The separation step can include multiple rinses or washes. For example, when the solid substrate is a microtiter plate, the wells may be washed several times with a washing solution, which typically includes those components of the incubation mixture that do not participate in specific bindings such as salts, buffer, detergent, non-specific protein, etc. Where the solid substrate is a magnetic bead, the beads may be washed one or more times with a washing solution and isolated using a magnet.

In some instances the assay is an ATPase assay. For example, a known amount of p97 can be added to a specified amount of assay substrate/buffer mix. The assay substrate/buffer mix can contain a candidate p97 inhibitor along with ATP, magnesium and other components. After mixing and incubation at an appropriate temperature (e.g., 20° C. to 42° C., preferably about 37° C.) for an appropriate time (e.g., 5 to 60 minutes, preferably about 15 minutes), the reaction is stopped, and the amount of ADP, ATP, and/or inorganic phosphate is measured. For example, ATP hydrolysis can be measured using a colorimetric ATPase assay kit such as those available from Innova Biosciences.

A plurality of assay mixtures can be run in parallel or series with different drug concentrations to obtain a different response to the various concentrations. One of these concentrations can serve as a negative control, for example, a zero drug concentration or a drug concentration below the limits of assay detection. For example, the binding and/or ATPase activity of wild type p97 can serve as a positive control against which the binding or activity of the mutant p97 can be measured under similar conditions. The binding and/or ATPase activity of a wild type p97 protein can, for example, be compared to a mutant p97 protein in the presence or absence of a candidate p97 inhibitor.

### Detecting Drug Resistance in Samples

The invention relates to detection of drug resistance in samples of cells and tissues from organisms, animals and human patients (e.g. subjects) suspected of exhibiting drug resistance. The present invention also relates to methods for evaluating the effectiveness of a drug for a subject such as a patient. Drug resistance or drug effectiveness can be evaluated in a sample taken from a subject. Such samples can be from a subject such as a mammal or human patient suspected of being resistant to a drug such as a p97 inhibitor. The methods can include detecting whether at least one mutation is present in a p97 nucleic acid or a p97 protein present in a sample. The presence of one or more mutations often correlates with a fold change in susceptibility or resistance of a subject, or a p97 protein, to a p97 inhibitor. The effectiveness of p97 inhibitor in the presence of at least one of such mutations can also be determined using, for example, enzymatic, phenotypic and genotypic methods such as those described herein.

Drug resistance in a subject can be determined using a method that includes determining whether at least one mutation is present in a p97 nucleic acid or a p97 protein present in a sample from the subject. The mutation can be any of the mutations described herein for a p97 polypeptide or a p97 nucleic acid. The mutation can be detected in one or more cells present in a sample from a subject, or a p97 nucleic acid or a p97 protein in a sample from the subject.

The sample can be collected from a subject suspected of having drug resistance. While the sample to be evaluated can be a bodily fluid such as blood, serum, plasma, saliva, urine, or a tissue sample ideally the sample will be a tissue biopsy of a tumor

Drug resistance is detected by determining that at least one mutation is present in a p97 nucleic acid or a p97 protein within the sample from a subject. Similarly, the effectiveness of a p97 inhibitor as a therapeutic agent can be evaluated by determining whether at least one mutation is present in a p97 nucleic acid or a p97 protein within the sample.

When at least one mutation is present in a p97 nucleic acid or a p97 protein within the sample, an alternative therapeutic regiment can be employed for the subject from whom the sample was obtained. For example, drug resistance to one type of drug (e.g., one type of p97 inhibitor) can be obviated or treated by employing a different drug (e.g., a different p97 inhibitor) as a therapeutic agent.

The p97 mutation can be, for example, any of the mutations described herein.

The mutation can be detected in a nucleic acid or a protein or a protein activity within a sample. The nucleic acids and/or proteins can be within impure samples (e.g., an unpurified bodily fluid or biopsy sample comprising both tumor and normal tissues), or within a purified sample. For example, the nucleic acids or proteins can be extracted, purified, and/or semi-purified. The nucleic acids within the samples can be DNA or RNA. Mutations within the nucleic acids can include modifications, single nucleotide polymorphisms (SNPs) and mutations (e.g., missense, nonsense, insertions, deletions, duplications).

Methods for the detection and diagnosis drug resistance can include use of any available methods for detecting mutations in a nucleic acid or a protein. For example, the mutation can be detected by measuring the ATPase activity of proteins in the sample in the presence and/or absence of the drug to which the subject may have resistance. Assay procedures are described herein. In another example, the mutations in a nucleic acid or a protein can include use of a primer, probe, binding entity or antibody that can bind to a mutant p97 nucleic acid or a mutant p97 protein.

Probes, primers, and/or antibodies can be employed in nucleic acid sequencing, SNP assay, restriction fragment length polymorphism (RFLP) assay, cell sorting assay, Northern blotting, nuclease protection assay, RNA fingerprinting, polymerase chain reaction, ligase chain reaction, Qbeta replicase, isothermal amplification method, strand displacement amplification, transcription based amplification systems, quantitative nucleic acid amplification assays (e.g., polymerase chain reaction assays), combined reverse transcription/nucleic acid amplification, nuclease protection (SI nuclease or RNAse protection assays), Serial Analysis Gene Expression (SAGE), next generation sequencing, gene expression microarray, in situ hybridization, nucleic acid amplification, reverse transcription, polymerase chain reaction, quantitative real time polymerase chain reaction (qRT-PCR), transcriptome sequencing, RNA-seq, next generation sequencing, mass spectroscopy, immunoassays, and combinations thereof, combinations thereof and other techniques available to the skilled artisan.

Detecting mutations can routinely be accomplished using nucleic acid sequencing, primer extension and/or nucleic acid hybridization techniques (see, e.g., Sambrook et al. (MOLECULAR CLONING: A LABORATORY MANUAL, Cold Spring Harbor, N.Y. (1989); see also Sambrook et al., MOLECULAR CLONING: A LABORATORY MANUAL, Cold Spring Harbor, N.Y. (2001)).

\For example, SNP analytical methods can be employed that include oligonucleotide extension using a SNP-specific primer to generate an analyte mixture containing primer-extended oligonucleotide copies of the p97 nucleic segment of interest. The analyte mixture can be contacted with a probe that binds specifically to a p97 mutant nucleic acid. For example, a series of p97 probes can be employed, where the probes are present on an array, each probe type immobilized to a separate location on the array. When a primer-extended oligonucleotide copy of a p97 nucleic segment hybridizes to a probe, a signal can be emitted, and the presence of mutation in a p97 nucleic acid can thereby be detected.

One example of an SNP method can include the steps of: (a) providing a DNA sample; (b) amplifying a segment of a p97 nucleic acid that may contain a SNP using a primer pair; (c) performing an oligonucleotide extension using a SNP-specific primer to generate an analyte mixture comprising one or more copies of p97 allele segment(s); (d) contacting the analyte mixture with one or more probes that can specifically hybridize to one or more p97 nucleic acids that include a SNP; (e) washing unbound analyte mixture from the one or more probes; (d) detecting one or more p97 allele segment(s) hybridized to one or more probes; and (e) identifying which p97 SNP in the DNA sample.

Antibodies specific to mutant p97 proteins can be employed to detect the mutation(s). Such antibodies can be generated against mutant p97 proteins or p97 peptides that include tone or more mutations. Such antibodies can recognize and distinguish the abnormal forms of p97 even in the presence of the normal (wild type) forms of the p97 protein in the same sample.

When a p97 mutation is detected in a sample, the subject from whom the sample was obtained can be drug resistant. A therapeutic agent that is not drug to which the subject is resistant can be administered to the subject. Detection of a specific mutation that is correlated with resistance to a specific type of p97 inhibitor indicates that such a p97 inhibitor should no longer be administered to the subject. Instead, the subject can be administered a different kind of drug or a drug such as a p97 inhibitor to which the sample is not resistant. For example, as illustrated in the Examples described herein certain p97 mutations correlate with resistance to specific p97 inhibitors. When such a p97 mutation is detected a different p97 inhibitor can be administered to a subject with the p97 mutation.

“Treatment” or “treating” includes providing an active compound to a patient in an amount effective to measurably reduce any symptom of a disease or disorder responsive to p97 inhibition and/or endoplasmic reticulum (ER) stress induction caused by accumulation of mis-folded proteins. The disease or disorder responsive to p97 inhibition and/or ER stress induction can be, e.g., a disorder associated with undesired cell proliferation such as a cancer or a viral infection. Cancers for treatment include both solid and disseminated hematological cancers, for example multiple myeloma and mantle cell lymphoma (MCL), chronic lymphocytic leukemia (CLL), Acute myeloid leukemia (AML), T-cell leukemia, Burkitt's lymphoma, retinoblastoma, osteosarcoma, breast cancer, bladder cancer, prostate cancer, renal carcinoma, small-cell lung cancer, non-small-cell lung cancer, mucinous cancers and a cancer associated with viral infections, such as a cervical cancer associated with human papilloma virus. Viral infections include cytomegalovirus and retroviral infections, for example human immunodeficiency virus (HIV) infection.

“Treating” or “treatment” as used herein also refers to methods that result in an alleviation of symptoms associated with a disorder or disease, or inhibition of worsening of those symptoms, or inhibition of further progression of a disorder or a disease, or prevention or prophylaxis of the disease or disorder, or curing the disease or disorder. Such treatment can result in a reduction in tumor burden. Similarly, as used herein, an “effective amount” or a “therapeutically effective amount” of a compound of the invention refers to an amount of the compound that alleviates, in whole or in part, symptoms associated with the disorder or condition, or halts or slows further progression or worsening of those symptoms, or prevents or provides prophylaxis for the disorder or condition. The effective amount can, for example, result in a reduced tumor burden.

### p97 Inhibitors

The p97 inhibitors typically are small molecules that inhibit p97 in vitro and/or in vivo. Examples include classes of compounds disclosed in the following patents and published patent applications: U.S. Pat. Nos. 9,062,026; 8,865,708; 8,722,019; 8,637,560; 8,518,968; 8,273,700; Published U.S. Patent Application Nos. 2009/0253717; 2004/005022; and PCT published application Nos. WO 2015089218; WO 2014015291; WO 2011140527; WO 2011069039; WO 2009011910 and WO 2010003908. The disclosures of each and every one of these references are incorporated by reference herein as if all were fully and completely reproduced in this application.

As examples of the effectiveness of the methods, techniques and procedures of the invention, the compounds of the following list demonstrate inhibition of certain mutant p97 polypeptides and lack of inhibition against certain other mutant p97 polypeptides although all of these examples display significant inhibition of wild type p97 polypeptide.

| Compound | |
|---|---|
| Number | IUPAC |
| 2 | N-benzyl-2-(2-methoxy-1H-1,3-benzodiazol-1-yl)-5,6,7,8- |
| | tetrahydroquinazolin-4-amine |
| 3 | 1-[4-(benzylamino)-5H,6H,7H,8H-pyrido[4,3-d]pyrimidin- |
| | 2-yl]-2-methyl-1H-indole-4-carboxamide |
| 4 | 1-[4-(benzylamino)-5H,7H,8H-pyrano[4,3-d]pyrimidin- |
| | 2-yl]-2-methyl-1H-indole-4-carboxamide |
| 5 | 1-[4-(benzylamino)-5,6,7,8-tetrahydroquinazolin-2-yl]-2- |
| | methoxy-1H-1,3-benzodiazole-4-carboxamide |
| 6 | 2-[4-(aminomethyl)-2-methyl-1H-indol-1-yl]-N-benzyl- |
| | 5,6,7,8-tetrahydroquinazolin-4-amine |

### Compositions

Compounds (e.g., p97 inhibitors) identified as active against mutant p97 proteins can be administered in various forms, depending on the disorder to be treated and the age, condition, body weight of the patient and the wisdom of the patient's attending physician. The compounds may be administered as oral, parenteral, topical or inhaled formulations prepared by procedures available in the art. The pharmaceutical compositions useful for administration are disclosed and described in detail in the literature and patent documents disclosing p97 inhibitors. Aspects of the invention herein enable identification of such compounds that display p97 inhibition of mutant p97 polypeptides in vitro or/and in vivo. Screening of unknown and known compounds and screening of known compounds that inhibit wild type p97 polypeptide to establish their ability to inhibit resistant, mutant p97 polypeptides in vitro or/and in vivo can be accomplished using the techniques, methods and procedures described herein,

Pharmaceutical compositions incorporating such p97 inhibitory compounds can be formulated according to the techniques and methods described in the above cited patent references. These compositions may be formulated to be administered orally, intraperitoneally, intravenously or by any other appropriate route for introduction into the blood stream. Excipients, diluents, carriers, pH adjusting agents as described in the foregoing references may be incorporated as given therein. The disclosures of pharmaceutical compositions given in these references are incorporated herein by reference as if fully repeated herein.

In this context, a “therapeutically effective amount” of a pharmaceutical composition means an amount effective, when administered to a patient, to provide a therapeutic benefit such as an amelioration of symptoms, e.g., an amount effective to decrease the symptoms of a disease or disorder. In particular, a “therapeutically effective amount” refers to an amount effective, at dosages and for periods of time necessary, to achieve the desired therapeutic result such as a decrease in tumor burden. A therapeutically effective amount is also one in which any toxic or detrimental effects of compounds of the invention are outweighed by the therapeutically beneficial effects.

A “subject” as defined below preferably is a human cancer patient. Such human cancer patients will benefit from the methods, techniques, assays and screens described herein. Such patients may be efficiently and effectively treated with therapeutically effective amounts of p97 inhibitors selected according to the study of the p97 polypeptide or encoding polynucleotide of the patient's cancer cells and coordination of the study with appropriate p97 inhibitors shown to be effective inhibitors against the patient's particular make-up of his or her cancer cell p97 complex.

### Kits

Kits are also described here for use in the diagnostic and therapeutic applications described or suggested above. For example, the kits can include components for detecting drug resistance, for evaluating whether p97 mutations are present in nucleic acids present in a sample, for evaluating which p97 mutations are present in nucleic acids present in a sample, and for evaluating whether a different therapeutic regiment is appropriate (e.g., because drug resistance to a currently administered drug is detected). Such a kit can also contain drugs (e.g., p97 inhibitors) for treatment of a subject.

Kits are also described herein for identifying p97 inhibitors. Such kits can include components for evaluating the inhibition of wild type and/or mutant p97 protein activity, for example, in the presence of a candidate test compound. These kits can include any of the mutant p97 polypeptides described herein and/or any of the cell lines described herein that express mutant p97 polypeptides. The kits can also include controls, such as a sample of wild type p97 protein, or a population of cells that express wild type p97. Additional controls such as p97 inhibitors with known activities can also be included.

The kits can include a package that includes containers of compounds (e.g., p97 inhibitors), mutant p97 polypeptides, cell lines expressing mutant p97 polypeptides, wild type p97 polypeptide preparations, cell lines that express wild type p97 polypeptide, probes for p97 DNA and/or p97 RNA, binding entities (e.g., antibodies) for detection of p97 polypeptides, assay components, reagents and sampling devices. Such a package can be a box, a bag, a satchel, plastic carton (such as molded plastic or other clear packaging), wrapper (such as, a sealed or sealable plastic, paper, or metallic wrapper), or other container. In some examples, kit components will be enclosed in a single packaging unit, such as a box or other container, which packaging unit may have compartments into which one or more components of the kit can be placed.

In other examples, a kit includes one or more containers, for instance vials, tubes, and the like that can separately contain, for example, one or more p97 polypeptides (mutant or wild type), one or more cell lines that express one or more p97 polypeptides (mutant or wild type), one or more drugs or compounds, one or more nucleic acid probes, one or more primers, one or more binding entities (e.g., antibodies), one or more assay platforms (e.g., arrays of probes with or without primers that can bind to p97 nucleic acids), more or more labels, one or more reagents for detecting a probe, primer or antibody, as well as positive and/or negative control samples or solutions.

For example, at least one of the containers can include at least one binding entity or antibody that binds with specificity or selectivity to a p97 mutant or wild type protein. In another embodiment, one of the containers can include a nucleic acid probe or primer that selectively hybridizes to a mutant p97 nucleic acid or wild type p97 nucleic acid. The binding entities (e.g. antibodies), probes and primers are or can be detectably labeled. For example, the binding entities, probes and primers can be packaged separately from the labels, and the label can be added to the binding entities, probes and primers during or after performance of an assay for a p97 nucleic acid or a p97 protein.

Kits can also contain vials, needles, syringes, finger-prick devices, alcohol swabs, gauze squares, cotton balls, bandages, latex gloves, incubation trays with variable numbers of troughs, adhesive plate sealers, data reporting sheets, which may be useful for handling, collecting and/or processing biological samples. Kits may also optionally contain implements useful for introducing samples into an assay chamber or a cell capturing device, including, for example, droppers, Dispo-pipettes, capillary tubes, rubber bulbs (e.g., for capillary tubes), and the like. Other components can also be present in the kits such as disposal means for discarding used devices and/or other items used with the device (such as subject samples, etc.). Such disposal means can include, without limitation, containers that are capable of containing leakage from discarded materials, such as plastic, metal or other impermeable bags, boxes or containers.

The kits can include instructions for use of a sampling device. The kit can also include instructions for performing an assay such as an immunoassay, SNP assay, RFLP assay, cell sorting assay, Northern blotting, nuclease protection assay, RNA fingerprinting, polymerase chain reaction, ligase chain reaction, Qbeta replicase, isothermal amplification method, strand displacement amplification, transcription based amplification systems, quantitative nucleic acid amplification assays (e.g., polymerase chain reaction assays), combined reverse transcription/nucleic acid amplification, nuclease protection (SI nuclease or RNAse protection assays), Serial Analysis Gene Expression (SAGE), next generation sequencing, gene expression microarray, in situ hybridization, nucleic acid amplification, reverse transcription, polymerase chain reaction, quantitative real time polymerase chain reaction (qRT-PCR), and the like.

### DEFINITIONS

As used herein, the terms “polypeptides” and “proteins” are employed interchangeably. Such “polypeptides” and “proteins” can be wild type or mutant p97 polypeptides or proteins, as well as variants, or fragments or portions thereof.

The term “subject” as used herein includes all animals. For example, the subject can be a mammal, a human or nonhuman primate, a dog, a cat, a horse, a cow, other farm animals, or a rodent (e.g. mice, rats, guinea pig, etc.). The term “subject,” “patient” and “individual” are used interchangeably herein. The “subject” can be a human or non-human animal provided a compound as described herein. “Subject” includes a patient, wherein a patient is a human or non-human animal in need of medical treatment. Medical treatment can include treatment of an existing condition, such as a disease or disorder, prophylactic or preventative treatment, or diagnostic treatment. In some embodiments the patient is a human patient. In certain embodiments treatment is treatment of an existing condition such as cancer.

“Providing” means giving, administering, selling, distributing, transferring (for profit or not), manufacturing, compounding, or dispensing.

The term “about” as used herein, when referring to a numerical value or range, allows for a degree of variability in the value or range, for example, within 10%, or within 5% of a stated value or of a stated limit of a range.

All percent compositions are given as weight-percentages, unless otherwise stated.

The following non-limiting Examples illustrate materials and methods employed during the development of the invention, as well as some of the aspects of the invention.

### Example 1: Materials and Methods

This Example describes some of the materials and methods employed in the development of the invention.

### Generation of Resistant Cell Lines

Dose titration of test compounds were conducted in Hct116, DLD-1, or LoVo cell lines. In brief, cells were plated in 384-well plates and a selected candidate drug compound was added in a 10-point 2-fold serial dilution. Viability was then measured after 72 hrs of drug exposure, using the Cell Titer Glo kit (Promega). Data was fit to a four-parameter sigmoidal curve to determine compound doses that result in 50% decrease in viability (GI₅₀). Hct116 cells were plated at 50,000 cells per 15 cm dish and treated with GI₅₀-GI₉₀ doses of test compound for 2-4 weeks until individual colonies of cells were visible on the plate. Colonies were isolated and expanded in separate dishes in the absence of test compound for subsequent characterization.

### Characterization of Resistant Cell Lines

Parental cell lines as well as cell lines selected for resistance to test compounds were plated in 384 well plates and treated with test compound or other related or control compounds in a dose titration. GI₅₀ of each compound was calculated for both parental and resistant cell lines. Resistance was measured by dividing the GI₅₀ of the resistant cell lines by the GI₅₀ of the parental cell line after 72 hrs of incubation with various dose titrations of test compounds (example shown in table 1). A fold-change in GI₅₀ between the parental and resistant cell lines was calculated.

### Sequencing of Resistant Cell Lines

Total RNA was isolated from a pellet of 1×10⁶ to 1×10⁷ cells using the Ambion Purelink total RNA isolation kit (Life Technologies). cDNA was then made using a VILO cDNA synthesis kit (Life Technologies). A DNA segment encoding p97 was amplified from the cDNA samples using PCR with the following primers: TCTGGAGCCGATTCAAAAGG (SEQ ID NO:7) with GAGCGGCGCATTGTATCAC (SEQ ID NO:8); and/or GGGAATCTGAAGCTGCCAAAG (SEQ ID NO:9) with TGCTAGAGAGTTCATGACCTCGG (SEQ ID NO:10). The p97 gene was then sequenced using four overlapping Sanger sequencing reactions. Results were compared to the wild-type p97 gene sequence to identify nucleotide mutations.

### Purification of Mutant Proteins

Mutations in p97 identified in test compound-resistant cell lines were added to p97 containing expression vector using site directed mutagenesis. Proteins were expressed in the B121[DE3] strain of E. coli and purified using His-tag mediated nickel purification and gel filtration chromatography.

### Biochemical Determination of ATPase Activity of Mutant p97

ATPase activity of p97 was measured using an ATP regenerating enzymatic system that couples the conversion of phosphoenolpyruvate (PEP) to pyruvate by pyruvate kinase with ADP conversion to ATP. Pyruvate is then converted to lactate by lactate dehydrogenase, which is coupled to the conversion of NADH to NAD⁺. The loss of NADH is measured by a decrease in optical absorbance as 280 nm allowing for real-time measurement of ATPase activity. Dose titrations of a test compound were added to the p97 ATPase assay and ATPase activity was monitored. The rate of ATPase activity was fit to four-parameter sigmoidal curve to calculate IC₅₀ of test compound. IC₅₀ values of test compounds for wild type p97 were compared to IC₅₀ values of mutant proteins to identify fold-changes in the potency of test compounds for mutant p97.

### Example 2: Drug Resistant p97 Cell Lines

This Example describes various p97 mutant cell lines that exhibit drug-resistance, and the p97 mutant proteins expressed by these cell lines.

Drug-resistant cell lines were generated as described in Example 1. In this example, the compound 2, 3, and 4 were used for generating drug resistant cell lines. Table 1 lists cell lines that are resistant to drug compounds 2, 3, and 4. Such cell lines have various p97 mutations, which are also identified in Table 1. Known compounds can be used as controls for assessing the relative inhibitory activity of the inventors' compounds. For example, compound 1 shown in Table 1 is Bortezomib, since Bortezomib does not target p97, little fold-change in GI₅₀ is seen for the p97 inhibitor resistant cell lines. Compounds 2, 3, 4, and 6 are all p97 inhibitors, therefore varying fold-change in GI₅₀ is seen when each resistant cell line is tested with such compounds.

TABLE 1
Nucleotide and amino acid mutations and cellular fold-change
in potency of test compounds for given cell lines.

| Cell Lines and Mutations | | | | | Compound Tested | | | | |
|---|---|---|---|---|---|---|---|---|---|
| | | Compound | | | In Resistant Line, | | | | |
| | | used to | | | Fold Change in GI50 Compared | | | | |
| | Cell | generate | Nucleotide | AA | to Parental Cell Line | | | | |
| Clone # | Line | resistance | Mutation | Mutation | 1 | 2 | 3 | 4 | 6 |
| 1 | Hct116 | 2 | G1945A | D649N | 1.0 | 4.5 | 22.6 | 20.0 | n.d. |
| 2 | Hct116 | 2 | T1421C | V474A | 1.0 | 5.5 | 11.9 | 8.7 | n.d. |
| 3 | Hct116 | 2 | G1442C | G481A | 0.9 | 5.4 | 27.6 | 19.4 | n.d. |
| 5 | Hct116 | 2 | A1946C | D649A | 1.0 | 7.3 | 21.3 | 18.7 | n.d. |
| 6 | Hct116 | 2 | G1582 | A528T | 1.1 | 5.0 | 13.4 | 11.8 | n.d. |
| 7 | Hct116 | 3 | G1975A | A659T | 0.9 | 3.5 | 6.3 | 3.8 | n.d. |
| 8 | Hct116 | 3 | A1978G | N660D | 0.8 | 1.8 | 2.5 | 1.8 | n.d. |
| 11 | Hct116 | 3 | G1410T | E470D | 1.0 | 4.0 | 5.0 | 5.4 | n.d. |
| 22 | Hct116 | 4 | A1978G | N660D | 1.0 | 3.1 | 6.8 | 3.1 | 1.8 |
| 25 | Hct116 | 4 | C1498A | P500T | 1.3 | 1.9 | 2.2 | 1.6 | 1.9 |
| 27 | Hct116 | 4 | A2062G | T688A | 1.1 | 1.6 | 50.3 | 50.3 | 2.3 |
| 29 | Hct116 | 4 | C1980A | N660K | 0.9 | 3.4 | 19.3 | 16.5 | 3.8 |
| 30 | Hct116 | 4 | T1546C | F516L | 0.9 | 5.4 | 10.0 | 7.1 | 4.1 |
| 31 | Hct116 | 4 | G1604A | C535Y | 0.9 | 3.0 | 6.4 | 4.6 | 3.3 |
| 33 | Hct116 | 4 | C1415T | P472L | 0.9 | 5.8 | 11.6 | 11.6 | 5.3 |
| 34 | Hct116 | 4 | G1410T/G | E470D/E | 0.8 | 3.0 | 3.8 | 3.7 | 2.7 |
| 42 | Hct116 | 4 | C1414G | P472A | 1.0 | 3.6 | 7.0 | 6.1 | 3.5 |
| 45 | Hct116 | 4 | T1615A | F539I | 0.8 | 3.1 | 6.9 | 6.4 | 3.4 |
| 47 | Hct116 | 4 | G1494T | E498D | 0.8 | 1.8 | 2.6 | 2.4 | 1.8 |
| 58 | DLD-1 | 4 | G1945G/A | D649D/N | 1.3 | 1.4 | 2.0 | 1.8 | 2.0 |
| 61 | DLD-1 | 4 | T1859T/C | I620I/L | 0.8 | 1.1 | 1.0 | 1.3 | 3.3 |
| 64 | DLD-1 | 4 | A1979G/A | N660N/S | 0.7 | 0.6 | 1.2 | 1.2 | 2.7 |
| 82 | DLD-1 | 4 | G1705G/A | A569A/T | 1.5 | 2.8 | 4.9 | 6.6 | 4.5 |
| 83 | DLD-1 | 4 | A1978G/A | N660N/D | 1.2 | 2.6 | 2.4 | 2.7 | 1.3 |
| 87 | DLD-1 | 4 | T1621T/C | S541P | 1.1 | 1.3 | 34.6 | 39.8 | 0.9 |
| 90 | Hct116 | 4 | A1418C | Q473P | 1.2 | 1.9 | 2.5 | 3.0 | 2.1 |
| 108 | Hct116 | 4 | C1936A | P646T | 0.9 | 1.3 | 1.6 | 2.0 | 1.6 |
| 117 | Hct116 | 4 | C1444A | L482I | 0.8 | 2.6 | 4.4 | 3.2 | 2.3 |
| 137 | Hct116 | 4 | C1424T | T475I | 0.8 | 1.6 | 2.6 | 4.4 | 2.8 |
| 139 | Hct116 | 4 | C1414T | P472S | 1.0 | 2.8 | 4.2 | 4.8 | 3.5 |
| 152 | Hct116 | 4 | C2063T | T688I | 1.0 | 2.1 | 40.2 | 60.7 | 1.4 |
| 180 | Hct116 | 4 | G1705A | A569T | 0.8 | 2.7 | 5.1 | 5.9 | 3.4 |
| 207 | Hct116 | 4 | T1454A | V485D | 1.0 | 7.0 | 27.9 | 17.0 | 2.7 |
| 221 | LoVo | 4 | T1421C/T | V474V/A | 0.9 | 2.2 | 4.1 | 2.2 | 2.2 |

The compound numbers listed in Table 1 correspond to the following compounds.

| Compound | |
|---|---|
| Number | IUPAC |
| 1 | [(1R)-3-methyl-1-[(2S)-3-phenyl-2-(pyrazin-2- |
| | ylformamido)propanamido]butyl]boronic acid |
| 2 | N-benzyl-2-(2-methoxy-1H-1,3-benzodiazol-1-yl)-5,6,7,8- |
| | tetrahydroquinazolin-4-amine |
| 3 | 1-[4-(benzylamino)-5H,6H,7H,8H-pyrido[4,3-d]pyrimidin- |
| | 2-yl]-2-methyl-1H-indole-4-carboxamide |
| 4 | 1-[4-(benzylamino)-5H,7H,8H-pyrano[4,3-d]pyrimidin-2- |
| | yl]-2-methyl-1H-indole-4-carboxamide |
| 5 | 1-[4-(benzylamino)-5,6,7,8-tetrahydroquinazolin-2-yl]-2- |
| | methoxy-1H-1,3-benzodiazole-4-carboxamide |
| 6 | 2-[4-(aminomethyl)-2-methyl-1H-indol-1-yl]-N-benzyl- |
| | 5,6,7,8-tetrahydroquinazolin-4-amine |

The difference in fold change of GI₅₀ values between test compounds can be the selection criteria for selecting a suitable drug for treatment of drug resistant patients. For example, as shown in Table 1, cell line #152 was generated by exposure to compound 4, and has a 60.7 fold change in resistance to compound 4 compared to the parental cell line. However, cell line #152 has only a 1.4 fold change in GI₅₀ for compound 6 when compared to the parental cell line. Therefore compound 6 may be a suitable drug for treatment of patients who become resistant to compound 4.

### Example 3: Drug Resistant p97 Proteins

This Example describes various p97 mutant recombinant proteins that exhibit drug-resistance.

Recombinant p97 protein containing resistant mutants were tested with compounds 2, 3, 4 and 5 in a biochemical ATPase assay.

Table 2 lists fold changes in IC₅₀ values for mutant proteins versus wild type protein for each test compound.

TABLE 2
Biochemical fold-change in potency of test compounds 2, 3, 4 and 5
for p97 with the indicated amino acid mutations

Fold Change in IC50
compared to WT Protein

| Mutation | 2 | 3 | 4 | 5 |
|---|---|---|---|---|
| E470D | 3.1 | 6.4 | 2.4 | 15.4 |
| Q473P | 1.6 | 2.5 | 1.8 | 0.3 |
| G481A | 1.9 | 8.2 | 2.7 | 4.1 |
| D649A | 2.8 | 7.0 | 7.6 | 67.0 |
| A659T | 2.8 | 18.2 | 6.1 | 110.0 |
| T688A | 8.8 | n. d. | 18.8 | 363.0 |

Differences in activity fold changes between test compounds for a given mutant p97 polypeptide indicates which test compound would be most effective for inhibiting that mutant p97 polypeptide. For example, test compound 5 has a 67.0 fold change in IC₅₀ for a p97 mutant polypeptide containing the D649A mutation, whereas test compound 2 has a 2.8 fold change. Lower IC₅₀ values and lower IC₅₀ fold changes indicate that a compound is more active than compounds with higher IC₅₀ values and higher IC₅₀ fold changes. Therefore, compound 2 would be a good drug candidate for treatment of subjects with resistant cancers harboring the D649A mutation in their p97 proteins.

### Embodiments and Specific Features of the Invention

Additional embodiments, details, characterizations and examples of the invention are provided by the following disclosure. These embodiments are numbered so that their combinations are clear and unambiguous.
1. A mutant p97 polypeptide comprising a sequence with at least 95% sequence identity to SEQ ID NO:1, 3, 4, 5, or 6 and with at least one amino acid difference compared to a human wild type p97 protein.
2. The mutant p97 polypeptide of embodiment 1 having at least one amino acid difference compared to a wild type p97 protein with SEQ ID NO:1. 3. The mutant p97 polypeptide of embodiment 1 or 2 having glycine at positions 480 and 481.
4. The mutant p97 polypeptide of any of embodiments 1-3 which does not have any of the following mutations: E305Q, E578Q, N348I, N624I, K251A, K524A, R359E, or R635E.
5. The mutant p97 polypeptide of any of embodiments 1-4 having at least one amino acid difference in an ATPase domain relative to a wild type p97 protein.
6. The mutant p97 polypeptide of any of embodiments 1-5 having at least one amino acid difference in its D2 domain, or within 15 amino acid positions of the D2 domain, compared to a wild type p97 protein D2 domain with SEQ ID NO:3 or 4.
7. The mutant p97 polypeptide of any of embodiments 1-6 having at least one amino acid difference compared to a wild type p97 protein segment with either of SEQ ID NO:4, 5, or 6.
8. The mutant p97 polypeptide of any of embodiments 1-7 comprising amino acid changes at one or more of the following positions: E470, P472, Q473, V474, T475, G481, L482, V485, E498, P500, F516, A528, C535, F539, S541, A569, I620, D649, D649, A659, N660, N660, T688, or any combination thereof.
9. The mutant p97 polypeptide of any of embodiments 1-8 comprising one or more of the following amino acid changes: E470D, E470E/D, P472A, P472L, P472S, Q473P, V474A, V474V/A, T475I, G481A, L482I, V485D, E498D, P500T, F516L, A528T, C535Y, F539I, S541P, A569A/T, A569T, I620I/L, P646T, D649A, D649D/N, D649N, A659T, N660D, N660K, N660N/D, N660N/S, T688A, T688I, or any combinations thereof.
10. The mutant p97 polypeptide of any of embodiments 1-9 with an ATPase activity that is not inhibited by one or more of the following compounds: N-benzyl-2-(2-methoxy-1H-1,3-benzodiazol-1-yl)-5,6,7,8-tetrahydroquinazolin-4-amine; 1-[4-(benzylamino)-5H,6H,7H,8H-pyrido[4,3-d]pyrimidin-2-yl]-2-methyl-1H-indole-4-carboxamide; 1-[4-(benzylamino)-5H,7H,8H-pyrano[4,3-d]pyrimidin-2-yl]-2-methyl-1H-indole-4-carboxamide; 1-[4-(benzylamino)-5,6,7,8-tetrahydroquinazolin-2-yl]-2-methoxy-1H-1,3-benzodiazole-4-carboxamide; 2-[4-(aminomethyl)-2-methyl-1H-indol-1-yl]-N-benzyl-5,6,7,8-tetrahydroquinazolin-4-amine; or a combination thereof.
11. A cell that expresses the mutant p97 polypeptide of any of embodiments 1-10.
12. The cell of embodiment 10, wherein the cell expresses the mutant p97 polypeptide from a heterologous promoter.
13. A cell population that expresses the mutant p97 polypeptide of any of embodiments 1-10.
14. The cell population of embodiment 13, wherein the cell expresses the mutant p97 polypeptide from a heterologous promoter.
15. A method comprising: contacting the mutant p97 polypeptide of any of embodiments 1-10 with a candidate test compound and determining the ATPase activity of the p97 polypeptide.
16. The method of embodiment 15, further comprising determining a concentration at which the candidate test compound inhibits 50% of the ATPase activity (IC₅₀) of the mutant p97 polypeptide.
17. The method of embodiment 15 or 16, further comprising determining a concentration at which the candidate test compound inhibits 50% of the ATPase activity (IC₅₀) of the mutant p97 polypeptide and comparing that IC₅₀ to a control IC₅₀ value.
18. The method of any of embodiments 15-17, wherein the ATPase activity of the mutant p97 polypeptide is compared to a control ATPase activity for a control p97 polypeptide.
19. The method of any of embodiments 15-18, wherein the ATPase activity of the mutant p97 polypeptide is compared to a control ATPase activity for a control p97 polypeptide, and wherein the control p97 polypeptide is a wild type p97 polypeptide or a second mutant p97 polypeptide with a different amino acid sequence than the p97 polypeptide.
20. The method of any of embodiments 15-19, wherein the mutant p97 polypeptide, the control p97 polypeptide and the second p97 polypeptide are expressed in cells.
21. A method comprising:

contacting a drug-resistant cell population with a candidate test compound; and

determining whether the candidate test compound inhibits cell growth of the cell population compared to a control drug-resistant cell population that is not contacted with the candidate test compound; wherein the drug-resistant cell population is resistant to a p97 inhibitor or chemotherapeutic agent.
22. The method of embodiment 21 wherein the determining step comprises determining a concentration at which the first p97 inhibitor inhibits 50% of cell population growth (GI50); or at which the candidate test compound inhibits 50% of cell population growth (GI50); and comparing the first p97 inhibitor GI50 or the candidate test compound GI50 to a control GI50 value.
23. The method of embodiment 21 or 22, wherein the drug-resistant cell population is resistant to one or more of the following compounds: N-benzyl-2-(2-methoxy-1H-1,3-benzodiazol-1-yl)-5,6,7,8-tetrahydroquinazolin-4-amine; 1-[4-(benzylamino)-5H,6H,7H,8H-pyrido[4,3-d]pyrimidin-2-yl]-2-methyl-1H-indole-4-carboxamide; 1-[4-(benzylamino)-5H,7H,8H-pyrano[4,3-d]pyrimidin-2-yl]-2-methyl-1H-indole-4-carboxamide; 1-[4-(benzylamino)-5,6,7,8-tetrahydroquinazolin-2-yl]-2-methoxy-1H-1,3-benzodiazole-4-carboxamide; 2-[4-(aminomethyl)-2-methyl-1H-indol-1-yl]-N-benzyl-5,6,7,8-tetrahydroquinazolin-4-amine; or a combination thereof.
24. The method of any of embodiments 21-23, wherein drug-resistant cell population expresses a mutant p97 polypeptide.
25. The method of embodiment 24, wherein the mutant p97 polypeptide has at least one amino acid difference in its ATPase domain relative to a wild type p97 protein.
26. The method of embodiment 24 or 25, wherein the mutant p97 polypeptide has at least one amino acid difference compared to a wild type p97 polypeptide with SEQ ID NO:1.
27. The method of any of embodiments 21-26, wherein the mutant p97 polypeptide has glycine at positions 480 and 481.
28. The method of any of embodiments 24-27, wherein the mutant p97 polypeptide does not have any of the following mutations E305Q, E578Q, N348I, N624I, K251A, K524A, R359E, or R635E.
29. The method of any of embodiments 24-28, wherein the mutant p97 polypeptide has at least one amino acid difference in its ATPase domain relative to a wild type p97 protein.
30. The method of any of embodiments 24-29, wherein the mutant p97 polypeptide has at least one amino acid difference in its D2 domain, or within 15 amino acid positions of the D2 domain, compared to a wild type p97 protein D2 domain with SEQ ID NO:3 or 4.
31. The method of any of embodiments 24-30, wherein the mutant p97 polypeptide has at least one amino acid difference compared to a wild type p97 protein segment with either of SEQ ID NO:4, 5, or 6.
32. The method of any of embodiments 24-31, wherein the mutant p97 polypeptide comprises one or more amino acid changes at any of the following positions of SEQ ID NO:1, 3, or 4: E470, P472, Q473, V474, T475, G481, L482, V485, E498, P500, F516, A528, C535, F539, S541, A569, I620, D649, D649, A659, N660, N660, T688, or any combination thereof.
33. The method of any of embodiments 24-32, wherein the mutant p97 polypeptide comprises one or more of the following amino acid changes compared to wild type: E470D, E470E/D, P472A, P472L, P472S, Q473P, V474A, V474V/A, T475I, G481A, L482I, V485D, E498D, P500T, F516L, A528T, C535Y, F539I, S541P, A569A/T, A569T, I620I/L, P646T, D649A, D649D/N, D649N, A659T, N660D, N660K, N660N/D, N660N/S, T688A, T688I, and combinations thereof.
34. The method of any of embodiments 21-33, wherein the drug-resistant cell population is a cancer cell population.
35. The method of any of embodiments 21-34, wherein determining whether the candidate test compound inhibits cell growth comprises determining a concentration at which the test compound inhibits 50% of growth (GI₅₀) of the chemotherapeutic drug-resistant cell population.
36. The method of any of embodiments 21-35, wherein determining whether the candidate test compound inhibits cell growth comprises comparing an GI₅₀ value of the candidate test compound for a cell population that expresses a mutant p97 polypeptide with an GI₅₀ value of the candidate test compound for a cell population that expresses a wild type p97 protein, after both mutant and wild type cell populations have been contacted with the candidate test compound.
37. The method of any of embodiments 21-36, wherein a candidate test compound can inhibit a mutant cell population growth when the candidate test compound has an GI₅₀ that is 2-fold less than the candidate test compound GI₅₀ for a parental cell line population from which mutant cell population was derived.
38. A method comprising determining whether a test sample comprises
   (i) a mutant p97 polypeptide, or
   (ii) a nucleic acid encoding the mutant p97 polypeptide, wherein the mutant p97 polypeptide comprises an amino acid sequence with one or more of amino acid changes at the following positions compared to a wild type p97 with SEQ ID NO:1, 3 or 4: E470, P472, Q473, V474, T475, G481, L482, V485, E498, P500, F516, A528, C535, F539, S541, A569, I620, P646, D649, D649, A659, N660, T688, and combinations thereof.
39. The method of embodiment 38, wherein the test sample is from a subject who is insensitive to a chemotherapeutic drug.
40. The method of embodiment 38 or 39, wherein the test sample is from a subject who is insensitive to a p97 inhibitor.
41. The method of any of any of embodiments 38-40, wherein the test sample is from a subject who is insensitive to one or more of the following compounds: N-benzyl-2-(2-methoxy-1H-1,3-benzodiazol-1-yl)-5,6,7,8-tetrahydroquinazolin-4-amine; 1-[4-(benzylamino)-5H,6H,7H,8H-pyrido[4,3-d]pyrimidin-2-yl]-2-methyl-1H-indole-4-carboxamide; 1-[4-(benzylamino)-5H,7H,8H-pyrano[4,3-d]pyrimidin-2-yl]-2-methyl-1H-indole-4-carboxamide; 1-[4-(benzylamino)-5,6,7,8-tetrahydroquinazolin-2-yl]-2-methoxy-1H-1,3-benzodiazole-4-carboxamide; 2-[4-(aminomethyl)-2-methyl-1H-indol-1-yl]-N-benzyl-5,6,7,8-tetrahydroquinazolin-4-amine; or a combination thereof.
42. The method of any of embodiments 38-41, further comprising administering a chemotherapeutic drug to a subject from whom the test sample was obtained when the subject is insensitive to a chemotherapeutic drug.
43. The method of any of embodiments 38-42, further comprising administering a p97 inhibitor to a subject from whom the test sample was obtained when the subject is insensitive to a chemotherapeutic drug.
44. The method of any of embodiments 38-43, further comprising administering a second p97 inhibitor to a subject from whom the test sample was obtained when the subject is insensitive to a first p97 inhibitor.
45. The method of any of embodiments 38-44, wherein the determining step comprises an ATPase assay, immunoassay, nucleic acid sequencing, SNP assay, restriction fragment length polymorphism (RFLP) assay, cell sorting assay, Northern blotting, nuclease protection assay, RNA fingerprinting, polymerase chain reaction, ligase chain reaction, Qbeta replicase, isothermal amplification method, strand displacement amplification, transcription based amplification systems, quantitative nucleic acid amplification assays (e.g., polymerase chain reaction assays), combined reverse transcription/nucleic acid amplification, nuclease protection (SI nuclease or RNAse protection assays), Serial Analysis Gene Expression (SAGE), next generation sequencing, gene expression microarray, in situ hybridization, nucleic acid amplification, reverse transcription, polymerase chain reaction, quantitative real time polymerase chain reaction (qRT-PCR), mass spectroscopy, cell sorting assay, immunoassay and combinations thereof.
46. A kit for detection of a p97 protein or a p97 nucleic acid and instructions for detecting a p97 protein or a p97 nucleic acid.
47. The kit of embodiment 46, wherein the p97 protein is one or more of the mutant p97 polypeptides of any of embodiments 1-10.
48. The kit of embodiment 46 or 47, wherein the p97 protein or the p97 nucleic acid is present in a sample from a subject.
49. The kit of any of embodiments 45-48, further comprising a sampling device.
50. The kit of any of embodiments 45-49, comprising assay components for performing an ATPase assay, immunoassay, nucleic acid sequencing, SNP assay, restriction fragment length polymorphism (RFLP) assay, cell sorting assay, Northern blotting, nuclease protection assay, RNA fingerprinting, polymerase chain reaction, ligase chain reaction, Qbeta replicase, isothermal amplification method, strand displacement amplification, transcription based amplification systems, quantitative nucleic acid amplification assays (e.g., polymerase chain reaction assays), combined reverse transcription/nucleic acid amplification, nuclease protection (SI nuclease or RNAse protection assays), Serial Analysis Gene Expression (SAGE), next generation sequencing, gene expression microarray, in situ hybridization, nucleic acid amplification, reverse transcription, polymerase chain reaction, quantitative real time polymerase chain reaction (qRT-PCR), mass spectroscopy, and combinations thereof.
51. A method of identifying a drug that is effective in drug-resistant cells comprising:
   contacting the chemotherapeutic drug-resistant cell population with a candidate test compound; and
   determining whether the candidate test compound inhibits p97 expression or activity in the cell population compared to a control drug-resistant cell population that is not contacted with the candidate test compound.
52. A method of identifying a drug that is effective in drug-resistant cells comprising:
   (a) contacting a cell population with a drug for a time and at a drug concentration sufficient to generate at least one drug resistant cell;
   (b) identifying a mutation in at least one drug resistant cell's genome; and
   (c) identifying a drug that is effective in at least one drug resistant cell.
53. The method of embodiment 52, further comprising isolating a mutant nucleic acid that encodes a mutant polypeptide from at least one drug resistant cell.
54. The method of embodiment 52 or 53, wherein identifying a mutation in at least one drug resistant cell's genome comprises primer extending, nucleic acid amplification, sequencing, performing SNP analysis, or a combination thereof, on one or more segments of at least one drug resistant cell's genome.
55. The method of any of embodiments 52-53, further comprising isolating a mutant polypeptide from at least one drug resistant cell.
56. The method of any of embodiments 52-55, further comprising synthesizing a mutant polypeptide from a mutant nucleic acid obtained from at least one drug resistant cell.
57. The method of any of embodiments 52-56, wherein identifying a drug that is effective in at least one drug resistant cell comprises: one or more separate enzymatic assays of a mutant polypeptide in the presence and/or absence of one or more candidate drug compounds, wherein the mutant polypeptide is isolated from at least one drug resistant cell, or wherein the mutant polypeptide is synthesized from a mutant nucleic acid obtained from at least one drug resistant cell.
58. The method of any of embodiments 52-57, wherein identifying a drug that is effective in at least one drug resistant cell comprises: contacting at least one drug resistant cell with a candidate drug compound and determining whether the cell is sensitive or resistant to the candidate drug compound.
59. The method of any of embodiments 52-58, wherein identifying a drug that is effective in at least one drug resistant cell comprises: contacting at least one drug resistant cell with a candidate drug compound and determining whether the cell growth of the at least one drug resistant cell.
60. A kit for identifying a drug that is effective in drug-resistant cells comprising any of the mutant p97 polypeptides of any of embodiments 1-10 separately packaged, any of the cells of embodiment 11, or a combination thereof.
61. The kit of embodiment 60, further comprising a control p97 polypeptide or a control cell line that expresses a control p97 polypeptide.
62. The kit of embodiment 60 or 61, further comprising one or more control compounds with known p97 inhibitory activities.
63. A method according to embodiment 38 wherein the p97 polypeptide does not have any of the following mutations: G480P, G481P, E305Q, E578Q, N348I, N624I, K251A, K524A, R359E, or R635E;
64. The method according to embodiment 44, wherein the second p97 inhibitor inhibits cell growth at a lower concentration than the first p97 inhibitor; or wherein the second p97 inhibitor inhibits ATPase activity at a lower concentration than the first p97 inhibitor.
65. The method embodiment 17, wherein the control IC₅₀ is: (i) the IC₅₀ value for a control p97 inhibitor; (ii) the IC₅₀ value for a wild type p97 polypeptide; (iii) the IC₅₀ value of a selected p97 inhibitor (e.g. a first or second p97 inhibitor) for a selected p97 polypeptide (e.g., a wild type or parental p97 polypeptide); (iv) the IC₅₀ value of the candidate test compound for a mutant or wild type p97 polypeptide with a different amino acid sequence than the p97 polypeptide; or (iv) a combination thereof.
66. The method of embodiment 22 wherein the control GI₅₀ value is: (i) the GI₅₀ value of a control compound for the cell population; (ii) the GI₅₀ value of the first p97 inhibitor or the GI₅₀ value of the candidate test compound for wild type cell population growth; (iii) the GI₅₀ value of the first p97 inhibitor or the GI₅₀ value of the candidate test compound for a mutant cell population that expresses a mutant p97 polypeptide with a different amino acid sequence than the p97 polypeptide; or (iv) a combination thereof.
67. The cell according to embodiment 11 wherein the mutant p97 polypeptide is recombinantly expressed from an expression cassette or expression vector within the cell.
68. A kit according to any one of embodiments 46-50 wherein the kit comprises probes, primers, or binding entities that can bind to the p97 nucleic acid or the p97 polypeptide.
69. The kit of embodiment 69 wherein the probes, primers, or binding entities can bind to a p97 polypeptide comprising a sequence with at least one amino acid difference compared to a wild type p97.
70. A cell or cell population of any one of claims 11-14 wherein the cell is a progeny of an established cancer cell line.
71. A cell or cell population of embodiment 70 wherein the cell line is an Hct116, DLD-1, or LoVo cell line.
72. A method, cell line, drug resistant cell line or mutant p97 polypeptide as set forth in any of embodiments 1, 15, 21, 38 and 51 and any embodiments dependent thereon, in which the test sample, cell line, drug resistant cell line or mutant p97 polypeptide has been produced by exposure of a wild type cellular sample or parent cell line or human patient to a p97 inhibitor or other drug that interacts with p97 polypeptide, allowing the exposed cellular sample or parent cell line to replicate or continuing inhibitor administration to the human patient and selecting surviving progeny of the cellular sample, patent cell line, or cancer cells of the human patient.
73. A method, cell line, drug resistant cell line or mutant p97 polypeptide that involves or is produced by a human cancer patient or is produce from a cell line useful for assay of p97 activity.

### REFERENCES

Brown et al., US20090253717, Inhibitors of p97.
Deshaies et al., WO2011140527, Methods and Compositions for Inhibition of the Transitional Endoplasmic Reticulum ATPase.
Huang et al., Interprotomer motion-transmission mechanism for the hexameric AAA ATPase p9′7, PNAS (2012).
Wacker et al., Using transcriptome sequencing to identify mechanisms of drug action and resistance, Nature Chem. Biol. 8: 235 (March 2012).
U.S. Pat. No. 8,518,968 by Weistner
Young et al., WO2010003908, Screening Assay for Compounds Targeting the p97 AAA-ATPase Complex in the Ubiquitin Proteasome System.

### General Statements

All patents and publications referenced or mentioned herein are indicative of the levels of skill of those skilled in the art to which the invention pertains, and each such referenced patent or publication is hereby specifically incorporated by reference to the same extent as if it had been incorporated by reference in its entirety individually or set forth herein in its entirety. Applicants reserve the right to physically incorporate into this specification any and all materials and information from any such cited patents or publications.

The inventions, examples, biological assays and results described and claimed herein have may attributes and embodiments include, but not limited to, those set forth or described or referenced in this application.

All patents, publications, scientific articles, web sites and other documents and references or mentioned herein are indicative of the levels of skill of those skilled in the art to which the invention pertains, and each such referenced document and material is hereby incorporated by reference to the same extent as if it had been incorporated verbatim and set forth in its entirety herein. The right is reserved to physically incorporate into this specification any and all materials and information from any such paten, publication, scientific article, web site, electronically available information, text book or other referenced material or document.

The written description of this patent application includes all claims. All claims including all original claims are hereby incorporated by reference in their entirety into the written description portion of the specification and the right is reserved to physically incorporated into the written description or any other portion of the application any and all such claims. Thus, for example, under no circumstances may the patent be interpreted as allegedly not providing a written description for a claim on the assertion that the precise wording of the claim is not set forth in exact wording within the written description portion of the patent.

While the invention has been described in conjunction with the detailed description thereof, the foregoing description is intended to illustrate and not limit the scope of the invention, which is defined by the scope of the appended claims. Thus, from the foregoing, it will be appreciated that, although specific non-limiting embodiments of the invention have been described herein for the purpose of illustration, various modifications may be made without deviating from the spirit and scope of the invention. Other aspects, advantages, and modifications are within the scope of the following claims and the present invention is not limited except as by the appended claims.

The specific methods and polypeptides described herein are representative of preferred non-limiting embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, in non-limiting embodiments or examples of the present invention, the terms “comprising”, “including”, “containing”, etc. are to be read expansively and without limitation. The methods and processes illustratively described herein suitably may be practiced in differing orders of steps, and that they are not necessarily restricted to the orders of steps indicated herein or in the claims.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by various non-limiting embodiments and/or preferred non-limiting embodiments and optional features, any and all modifications and variations of the concepts herein disclosed that may be resorted to by those skilled in the art are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

It is also to be understood that as used herein and in the appended claims, the singular forms “a,” “an,” and “the” include plural reference unless the context clearly dictates otherwise, for example, the term “X and/or Y” means “X” or “Y” or both “X” and “Y”, and the letter “s” following a noun designates both the plural and singular forms of that noun. In addition, where features or aspects of the invention are described in terms of Markush groups or as alternatives indicated by the conjunction “or”, it is intended, and those skilled in the art will recognize, that the invention embraces and is also thereby described in terms of any individual member and any subgroup of members of the Markush group or alternatives listing, and the right is reserved to revise the application or claims to refer specifically to any individual member or any subgroup of members of the Markush group or alternatives listing. It is also understood that the phrases “at least one” and “one or more” mean: a) one of the members of a Markush group or alternatives list that follows these phrases, and b) multiples of the members of the Markush group or alternatives list including any combination of two or more up to all members of the Markush group or alternatives list.

The following claims summarize aspects of the invention.

1-29. (canceled)

30. A method for treatment of a patient suffering from cancer or viral disease comprising:
a) Obtaining from the patient a first test sample of cells from the cancer or cells infected with a virus;
b) Assaying one or more cells of the first sample to determine the peptide sequence of the p97 polypeptide of the one or more cells thereby providing a determined p97 polypeptide sequence;
c) Comparing the determined p97 peptide sequence with the sequences of wild type p97 peptides of SEQ ID NO:1, 2 or 4 and mutant p97 polypeptides having amino acid sequences with one or more of amino acid changes at the following positions of the wild type p97 peptides with SEQ ID NO:1, 3 or 4: E470, P472, Q473, V474, T475, G481, L482, V485, E498, P500, F516, A528, C535, F539, S541, A569, I620, P646, D649, D649, A659, N660, T688, E305Q, E578Q, N348I, N624I, K251A, K524A, R359E, R635E, E470D, E470E/D, P472A, P472L, P472S, Q473P, V474A, V474V/A, T475I, G481A, L482I, V485D, E498D, P500T, F516L, A528T, C535Y, F539I, S541P, A569A/T, A569T, I620I/L, P646T, D649A, D649D/N, D649N, A659T, N660D, N660K, N660N/D, N660N/S, T688A, T688I, G480P, G481P, E305Q, E578Q, N348I, N624I, K251A, K524A, R359E, R635E and combinations thereof.
d) Determining the sequence of the wild type p97 polypeptide or mutant p97 polypeptide that is at least 95 percent identical with the determined p97 peptide sequence thereby providing a first identified wild type or mutant p97 polypeptide;
e) Administering a first p97 inhibitor compound that inhibits at least about 20 percent ATPase activity of the first identified wild type or mutant p97 polypeptide.

31. A method according to claim 30 wherein the first p97 inhibitor compound is selected from compounds described in U.S. Pat. Nos. 9,062,026; 8,865,708; 8,722,019; 8,637,560; 8,518,968; 8,273,700; Published U.S. Patent Application Nos. 2009/0253717; 2004/005022; and PCT published application Nos. WO 2015089218; WO 2014015291; WO 2011140527; WO 2011069039; WO 2009011910 and WO 2010003908.

32. A method according to claim 31 wherein the assay of step b comprises an ATPase assay, immunoassay, nucleic acid sequencing, SNP assay, restriction fragment length polymorphism (RFLP) assay, cell sorting assay, Northern blotting, nuclease protection assay, RNA fingerprinting, polymerase chain reaction, ligase chain reaction; Qbeta replicase, isothermal amplification method, strand displacement amplification, transcription based amplification systems, quantitative nucleic acid amplification assays (e.g., polymerase chain reaction assays), combined reverse transcription/nucleic acid amplification, nuclease protection (SI nuclease or RNAse protection assays), Serial Analysis Gene Expression (SAGE), next generation sequencing, gene expression microarray, in situ hybridization, nucleic acid amplification, reverse transcription, polymerase chain reaction, quantitative real time polymerase chain reaction (qRT-PCR), mass spectroscopy, cell sorting assay, immunoassay and combinations thereof.

33. A method according to claim 32 further comprising:
a) Obtaining from the patient a second test sample of cells from the cancer or cells infected with the virus in the event that the first p97 inhibitor compound no longer inhibits the ATPase activity of the cells of the second test sample;
b) Conducting steps b, c, d of claim 30 upon the one or more cells of the second test sample to provide a second identified wild type or mutant p97 polypeptide;
c) Administering a second p97 inhibitor compound that inhibits at least about 20 percent ATPase activity of the second identified wild type or mutant p97 polypeptide.

34. A method according to claim 33 wherein the second p97 inhibitor compound is selected from compounds described in U.S. Pat. Nos. 9,062,026; 8,865,708; 8,722,019; 8,637,560; 8,518,968; 8,273,700; Published U.S. Patent Application Nos. 2009/0253717; 2004/005022; and PCT published application Nos. WO 2015089218; WO 2014015291; WO 2011140527; WO 2011069039; WO 2009011910 and WO 2010003908.

35. A method according to claim 34 wherein the assay of step b comprises an ATPase assay, immunoassay, nucleic acid sequencing, SNP assay, restriction fragment length polymorphism (RFLP) assay, cell sorting assay, Northern blotting, nuclease protection assay, RNA fingerprinting, polymerase chain reaction, ligase chain reaction; Qbeta replicase, isothermal amplification method, strand displacement amplification, transcription based amplification systems, quantitative nucleic acid amplification assays (e.g., polymerase chain reaction assays), combined reverse transcription/nucleic acid amplification, nuclease protection (SI nuclease or RNAse protection assays), Serial Analysis Gene Expression (SAGE), next generation sequencing, gene expression microarray, in situ hybridization, nucleic acid amplification, reverse transcription, polymerase chain reaction, quantitative real time polymerase chain reaction (qRT-PCR), mass spectroscopy, cell sorting assay, immunoassay and combinations thereof.

36. A method according to claim 30 further comprising:
a) Obtaining a candidate test compound from any one of the compounds disclosed in U.S. Pat. Nos. 9,062,026; 8,865,708; 8,722,019; 8,637,560; 8,518,968; 8,273,700; Published U.S. Patent Application Nos. 2009/0253717; 2004/005022; and PCT published application Nos. WO 2015089218; WO 2014015291; WO 2011140527; WO 2011069039; WO 2009011910 and WO 2010003908;
b) Determining a concentration at which the candidate test compound inhibits at least 20% of the ATPase activity (IC₅₀) of wild type p97 and mutant p97 polypeptides having amino acid sequences with one or more of amino acid changes at the following positions compared to a wild type p97 with SEQ ID NO:1, 3 or 4: E470, P472, Q473, V474, T475, G481, L482, V485, E498, P500, F516, A528, C535, F539, S541, A569, I620, P646, D649, D649, A659, N660, T688, E305Q, E578Q, N348I, N624I, K251A, K524A, R359E, R635E, E470D, E470E/D, P472A, P472L, P472S, Q473P, V474A, V474V/A, T475I, G481A, L482I, V485D, E498D, P500T, F516L, A528T, C535Y, F539I, S541P, A569A/T, A569T, I620I/L, P646T, D649A, D649D/N, D649N, A659T, N660D, N660K, N660N/D, N660N/S, T688A, T688I, G480P, G481P, E305Q, E578Q, N348I, N624I, K251A, K524A, R359E, R635E and combinations thereof;
c) Selecting any candidate test compound as a p97 inhibitor compound that displays at least about 20 percent inhibition of in vitro ATPase activity of any one of the wild type p97 and mutant p97 polypeptides at a therapeutically effective amount.

37. A method according to claim 36 wherein the therapeutically effective amount is in a range of about 10 nanomolar to about 1 micromolar.

38. A method according to claim 30 wherein the inhibition of the ATPase activity is at least about 50%.

39. A method according to claim 33 wherein the inhibition of the ATPase activity is at least about 50%.

40. A method according to claim 36 wherein the inhibition of the ATPase activity is at least about 50%.

41. A method according to claim 30 wherein the treatment is treatment of cancer and the cancer is a solid or disseminated hematological cancer, multiple myeloma and mantle cell lymphoma (MCL), chronic lymphocytic leukemia (CLL), acute myeloid leukemia (AML), T-cell leukemia, Burkitt's lymphoma, retinoblastoma, osteosarcoma, bladder cancer, renal carcinoma, small-cell lung cancer, a mucinous cancer, a multiple myeloma, a metastatic breast cancer or tumor, a non-small cell lung cancer or tumor, a prostate cancer or tumor, an advanced colorectal cancer or tumor, an ovarian cancer or tumor, primary peritoneal carcinoma, a hormone refractory prostate cancer or tumor, a squamous cell carcinoma of the head, a squamous cell carcinoma of the neck, a metastatic pancreatic adenocarcinoma, a gastroesophageal cancer or tumor, a gastrointestinal cancer or tumor, a stomach cancer or tumor, a leukemia, a non-Hodgkin's lymphoma, or combinations thereof.

42. A method according to claim 30 wherein the treatment is treatment of infection and the virus is human papilloma virus, cytomegalovirus or human immunodeficiency virus.


SEQUENCE LISTING (10 sequences)

SEQ ID NO: 1 - protein, 806 aa, Homo sapiens
MASGADSKGDDLSTAILKQKNRPNRLIVDEAINEDNSVVSLSQPKMDELQLFRGDTVLLKGKKRREAVCIVLSDDTCSDEKIRMNRVVRNNLRVRLGDVISIQPCPDVKYGKRIHVLPIDDTVEGITGNLFEVYLKPYFLEAYRPIRKGDIFLVRGGMRAVEFKVVETDPSPYCIVAPDTVIHCEGEPIKREDEEESLNEVGYDDIGGCRKQLAQIKEMVELPLRHPALFKAIGVKPPRGILLYGPPGTGKTLIARAVANETGAFFFLINGPEIMSKLAGESESNLRKAFEEAEKNAPAIIFIDELDAIAPKREKTHGEVERRIVSQLLTLMDGLKQRAHVIVMAATNRPNSIDPALRRFGRFDREVDIGIPDATGRLEILQIHTKNMKLADDVDLEQVANETHGHVGADLAALCSEAALQAIRKKMDLIDLEDETIDAEVMNSLAVTMDDFRWALSQSNPSALRETVVEVPQVTWEDIGGLEDVKRELQELVQYPVEHPDKFLKFGMTPSKGVLFYGPPGCGKTLLAKAIANECQANFISIKGPELLTMWFGESEANVREIFDKARQAAPCVLFFDELDSIAKARGGNIGDGGGAADRVINQILTEMDGMSTKKNVFIIGATNRPDIIDPAILRPGRLDQLIYIPLPDEKSRVAILKANLRKSPVAKDVDLEFLAKMTNGFSGADLTEICQRACKLAIRESIESEIRRERERQTNPSAMEVEEDDPVPEIRRDHFEEAMRFARRSVSDNDIRKYEMFAQTLQQSRGFGSFRFPSGNQGGAGPSQGSGGGTGGSVYTEDNDDDLYG

SEQ ID NO: 2 - DNA, 3859 bp, Homo sapiens
gtgatctgcgggttgctggggagaggcgcggagaggcgggcgagagtccgcagggcaggcgctgattggctgaggtgggagcagcttcccttccgatgattcggctcttctcggctcagtctcagcgaagcgtctgcgaccgtcgtttgagtcgtcgctgccgctgccgctgccactgccactgccacctcgcggatcaggagccagcgttgttcgcccgacgcctcgctgccggtgggaggaagcgagagggaagccgcttgcgggtttgtcgccgctgctcgcccaccgcctggaagagccgagccccggcccagtcggtcgcttgccaccgctcgtagccgttacccgcgggccgccacagccgccggccgggagaggcgcgcgccatggcttctggagccgattcaaaaggtgatgacctatcaacagccattctcaaacagaagaaccgtcccaatcggttaattgttgatgaagccatcaatgaggacaacagtgtggtgtccttgtcccagcccaagatggatgaattgcagttgttccgaggtgacacagtgttgctgaaaggaaagaagagacgagaagctgtttgcatcgtcctttctgatgatacttgttctgatgagaagattcggatgaatagagttgttcggaataaccttcgtgtacgcctaggggatgtcatcagcatccagccatgccctgatgtgaagtacggcaaacgtatccatgtgctgcccattgatgacacagtggaaggcattactggtaatctcttcgaggtataccttaagccgtacttcctggaagcgtatcgacccatccggaaaggagacatttttcttgtccgtggtgggatgcgtgctgtggagttcaaagtggtggaaacagatcctagcccttattgcattgttgctccagacacagtgatccactgcgaaggggagcctatcaaacgagaggatgaggaagagtccttgaatgaagtagggtatgatgacattggtggctgcaggaagcagctagctcagataaaggagatggtggaactgcccctgagacatcctgccctctttaaggcaattggtgtgaagcctcctagaggaatcctgctttacggacctcctggaacaggaaagaccctgattgctcgagctgtagcaaatgagactggagccttcttcttcttgatcaatggtcctgagatcatgagcaaattggctggtgagtctgagagcaaccttcgtaaagcctttgaggaggctgagaagaatgctcctgccatcatcttcattgatgagctagatgccatcgctcccaaaagagagaaaactcatggcgaggtggagcggcgcattgtatcacagttgttgaccctcatggatggcctaaagcagagggcacatgtgattgttatggcagcaaccaacagacccaacagcattgacccagctctacggcgatttggtcgctttgacagggaggtagatattggaattcctgatgctacaggacgcttagagattcttcagatccataccaagaacatgaagctggcagatgatgtggacctggaacaggtagccaatgagactcacgggcatgtgggtgctgacttagcagccctgtgctcagaggctgctctgcaagccatccgcaagaagatggatctcattgacctagaggatgagaccattgatgccgaggtcatgaactctctagcagttactatggatgacttccggtgggccttgagccagagtaacccatcagcactgcgggaaaccgtggtagaggtgccacaggtaacctgggaagacatcgggggcctagaggatgtcaaacgtgagctacaggagctggtccagtatcctgtggagcacccagacaaattcctgaagtttggcatgacaccttccaagggagttctgttctatggacctcctggctgtgggaaaactttgttggccaaagccattgctaatgaatgccaggccaacttcatctccatcaagggtcctgagctgctcaccatgtggtttggggagtctgaggccaatgtcagagaaatctttgacaaggcccgccaagctgccccctgtgtgctattctttgatgagctggattcgattgccaaggctcgtggaggtaacattggagatggtggtggggctgctgaccgagtcatcaaccagatcctgacagaaatggatggcatgtccacaaaaaaaaatgtgttcatcattggcgctaccaaccggcctgacatcattgatcctgccatcctcagacctggccgtcttgatcagctcatctacatcccacttcctgatgagaagtcccgtgttgccatcctcaaggctaacctgcgcaagtccccagttgccaaggatgtggacttggagttcctggctaaaatgactaatggcttctctggagctgacctgacagagatttgccagcgtgcttgcaagctggccatccgtgaatccatcgagagtgagattaggcgagaacgagagaggcagacaaacccatcagccatggaggtagaagaggatgatccagtgcctgagatccgtcgagatcactttgaagaagccatgcgctttgcgcgccgttctgtcagtgacaatgacattcggaagtatgagatgtttgcccagacccttcagcagagtcggggctttggcagcttcagattcccttcagggaaccagggtggagctggccccagtcagggcagtggaggcggcacaggtggcagtgtatacacagaagacaatgatgatgacctgtatggctaagtggtggtggccagcgtgcagtgagctggcctgcctggaccttgttccctgggggtgggggcgcttgcccaggagagggaccaggggtgcgcccacagcctgctccattctccagtctgaacagttcagctacagtctgactctggacagggggtttctgttgcaaaaatacaaaacaaaagcgataaaataaaagcgattttcatttggtaggcggagagtgaattaccaacagggaattgggccttgggcctatgccatttctgttgtagtttggggcagtgcaggggacctgtgtggggtgtgaaccaaggcactactgccacctgccacagtaaagcatctgcacttgactcaatgctgcccgagccctcccttccccctatccaacctgggtaggtgggtaggggccacagttgctggatgtttatatagagagtaggttgatttattttacatgcttttgagttaatgttggaaaactaatcacaagcagtttctaaaccaaaaaatgacatgttgtaaaaggacaataaacgttgggtcaaaatggagcctgagtcctgggccctgtgcctgcttcttttcctgggaacagccttgggctacccaccactcccaaggcattcttccaaatgtgaaatcctggaagtaagattgcaccttcttcctctcctgatcaacatcggtatgatgtctcctgttgcctcaccctttgtctgcagtatcactggataggactggtggaaagggagcagcctgacagagctccaaatgtggagaatatggcatccctccacctatatttgatgtggacggtaaggctaggcctgcaggatcccttatcctgaccaaagactgtgttggggtgccatttgaaaatcgcagggttgcaaaagaatacaatcttacttgcaggtggatattctctatactctcttttaatgcatctaaaaatcccaaacatcccctggttggtgatcacttacagttgtgtccacctttattttatgtactttgattaaaaaaaaaaaactttttgttaatat

SEQ ID NO: 3 - protein, 283 aa, Homo sapiens
GLEDVKRELQELVQYPVEHPDKFLKFGMTPSKGVLFYGPPGCGKTLLAKAIANECQANFISIKGPELLTMWFGESEANVREIFDKARQAAPCVLFFDELDSIAKARGGNIGDGGGAADRVINQILTEMDGMSTKKNVFIIGATNRPDIIDPAILRPGRLDQLIYIPLPDEKSRVAILKANLRKSPVAKDVDLEFLAKMTNGFSGADLTEICQRACKLAIRESIESEIRRERERQTNPSAMEVEEDDPVPEIRRDHFEEAMRFARRSVSDNDIRKYEMFAQTLQ

SEQ ID NO: 4 - protein, 210 aa, Homo sapiens
GLEDVKRELQELVQYPVEHPDKFLKFGMTPSKGVLFYGPPGCGKTLLAKAIANECQANFISIKGPELLTMWFGESEANVREIFDKARQAAPCVLFFDELDSIAKARGGNIGDGGGAADRVINQILTEMDGMSTKKNVFIIGATNRPDIIDPAILRPGRLDQLIYIPLPDEKSRVAILKANLRKSPVAKDVDLEFLAKMTNGFSGADLTEI

SEQ ID NO: 5 - protein, 40 aa, Homo sapiens
DEKSRVAILKANLRKSPVAKDVDLEFLAKMTNGFSGADLT

SEQ ID NO: 6 - protein, 64 aa, Homo sapiens
EVPQVTWEDIGGLEDVKRELQELVQYPVEHPDKFLKFGMTPSKGVLFYGPPGKTLLAKAIANEC

SEQ ID NO: 7 - DNA, 20 bp, Artificial Sequence
  A synthetic oligonucleotide
tctggagccgattcaaaagg

SEQ ID NO: 8 - DNA, 19 bp, Artificial Sequence
  A synthetic oligonucleotide
gagcggcgcattgtatcac

SEQ ID NO: 9 - DNA, 21 bp, Artificial Sequence
  A synthetic oligonucleotide
gggaatctgaagctgccaaag

SEQ ID NO: 10 - DNA, 23 bp, Artificial Sequence
  A synthetic oligonucleotide
tgctagagagttcatgacctcgg